(19) United States
(12) Patent Application Publication
(10) Pub. No.: US 2009/0214622 A1
(43) Pub. Date: Aug. 27, 2009

(54) Nanoporous Membrane and Method of Preparation Thereof

(76) Inventors: Gerard Eddy Poinern, Western Australia (AU); Fiona Melanie Wood, Western Australia (AU); Mark William Fear, Western Australia (AU); Leigh Gregory Parkinson, Western Australia (AU)

Correspondence Address: ROBINS & PASTERNAK, 1731 EMBARCADERO ROAD, SUITE 230, PALO ALTO, CA, 94303, US

(21) Appl. No.: 12/085,403

(22) PCT Filed: Nov. 24, 2006
(86) PCT No.: PCT/AU2006/001784

§ 371 Date: Feb. 17, 2009

(30) Foreign Application Priority Data
Nov. 25, 2005 (AU) 2005906583

(51) Int. Cl. A61K 9/70; C12M 3/00; C12N 5/06; A61K 35/12; A61P 17/02; B44C 1/22

(52) U.S. Cl. 424/443; 435/305.2; 435/401; 424/93.7; 216/83; 216/41

(57) ABSTRACT

A nanoporous membrane structure characterised in that it has a hexagonal array of tubes with a substantially uniform inter-pore distance between the tubes wherein the distance is within the range from 10-500 nm and wherein the tubes may have a depth of up to 500 μm.

### FIELD OF THE INVENTION

The present invention relates to the manufacture and use of nanoporous membrane structures. In particular, the present invention relates to the use of nanoporous membrane structures in the treatment of tissue damage, such as wound repair.

### BACKGROUND ART

With advancements in nanotechnology over recent years, many research groups have sought to fabricate uniform nanoscale structures. Many of the potential applications of nanotechnology would benefit from nanostructures of specific dimensions in ordered arrays. However, with the number of uncontrollable processes occurring at the nanoscale, there is a demand for a systematic approach for fabricating reproducible nano-materials.

If such a material can be prepared then the applications to which the material may be placed are many and varied. One such use would be in the field of tissue engineering. Tissue engineering is a technology concerned with the maintenance or improvement of tissue function in wound healing. One of the principal methods behind tissue engineering involves the in vitro seeding of human cells onto a biocompatible scaffold to provide support for cellular attachment, proliferation and differentiation to form new tissues in 3-dimensions (Chu et al. 2004). It is well established that substratum topography has direct effects on the behaviour of cells and significant research has focused on the fabrication of an ideal scaffold for the promotion of tissue growth (Flemming et al. 1999).

For a scaffold, pore size, porosity and surface area are generally recognised as important parameters, as cells utilise the additional surface area for attachment, and thrive in an environment where extracellular fluids can circulate freely (Darling & Sun 2003). Most scaffolds are fabricated from aliphatic polyesters such as polyglycolic acid (PGA), polylactic acid (PLLA) or their co-polymers (PGLA) and polycaprolactone (PCL).

Recently, the potential of nano-structured scaffolds has been considered for tissue engineering. The topography of basement membranes, which are found throughout the vertebrate body and serve as substrata for other cellular structures, are composed of extracellular components of nanometre sized dimensions. However, despite the recognition of the importance of substrate topography, relatively little is known about the effects of nanometre scale features on cell behaviour.

Nano-fibrous polymeric scaffold structures strive to mimic the extracellular matrix (ECM) component of collagen, which is known to exist in bundles which range from 50-500 nm in size. The use of nano-fibrous scaffolds is thought to enhance cell adhesion as it provides a higher surface-to-volume ratio of substrate for attachment. Since cell migration, proliferation and differentiated function are all dependent upon the initial adhesion, tissue growth is expected to be enhanced on these scaffolds. However, it has also been suggested that the porous structure in these woven matrices is derived from the interstitial space of randomly arranged filaments, which is not adequate for cellular proliferation (Chu et al. 2004).

Furthermore, concurrent research by Wan et al. (2005) confirmed that cell attachment and proliferation are highly dependent on the physical properties of a scaffold. Topographical structure can influence cell responses by a phenomenon known as “contact guidance”;—in which cells orientate themselves with the surface detail. It was shown that osteoblast cell (bone forming cells) adhesion was significantly promoted on nano-structured substrates, with higher attachment efficiency on the rough surfaces when compared to a controlled smooth surface. It was concluded that the combination of surface roughness and chemistry was a key point for promoting cell proliferation on the surface of a material. This was consistent with the earlier suggestion of Flemming et al. (1999) that substratum topography has direct effects on the ability of cells to orientate, migrate, and produce organised arrangements.

The random mesh-like structures of these micro- and nano-fibrous scaffolds aim to entrap cells in an attempt to organise them into specific shapes and sizes. However, the cells must eventually overcome the template to form new tissues and as proposed by Chu et al. (2004), the interstitial porous structure does not allow free proliferation of cells. It has also been suggested that cells may exhibit inflammatory responses to polymer scaffolds (as they overcome the polymer template and processes its degradation by-products), and as such, responses are an impediment to fast wound healing. As a result, there is a need for structures with nanometre-sized topographical detail to positively influence cell behaviour, and allow free cell interaction and proliferation.

### SUMMARY OF THE INVENTION

The subject invention relates to a unique nanoporous membrane structure and method for its preparation that is rapid, efficient and simple to prepare and apply. It also relates to a method for treating a patient using the unique nanoporous membrane structure. Use of the described nanoporous membrane structure has been found to reduce the complexity associated with the use of conventional scaffold structures.

According to a first aspect the invention provides a nanoporous membrane structure characterised in that it has a hexagonal array of tubes with a substantially uniform inter-pore distance between the tubes wherein the distance is within the range from 10-500 nm and wherein the tubes may have a depth of up to 500 μm. Preferably the inter-pore distance between tubes is within the range from 50-420 nm. The pore density is usually in the range from 10⁹ to 10¹² pores/cm². Preferably the tubes which open with the pores are substantially aligned perpendicular to the surface of the membrane.

According to a second aspect of the invention there is provided a method for preparing a nanoporous membrane, comprising the steps of:
a) preparing an aluminium film;
b) subjecting the aluminium film to a first anodising step;
c) subjecting the aluminium film to wet chemical etching;
d) subjecting the aluminium film to a second anodising step, wherein the conditions employed in this step are similar to or substantially the same as the conditions as used in the first anodising step.

Preferably the membrane is prepared using a method, comprising the steps of:
a) preparing an aluminium film;
b) subjecting the aluminium film to a first anodising step;
c) wet chemical etching the aluminium film;
d) subjecting the aluminium film to a second anodising step wherein the conditions employed in this step are similar to or substantially the same as the conditions as used in the first anodising step;
e) contacting the aluminium film from step (d) with a material capable of supporting the film;
f) suspending the aluminium film in a solution which completely removes the unprotected aluminium substrate; and
g) removing the layer of oxide to produce nano-channels throughout the alumina film.

According to a third aspect of the invention there is provided a nanoporous membrane structure, produced according to the above method.

According to a fourth aspect of the invention, there is provided a method for preparing a nanoporous membrane, comprising the steps of:
a) preparing an aluminium film;
b) applying a protective layer to one side of the aluminium film;
c) subjecting the aluminium film to a first anodising step;
d) subjecting the aluminium film to wet chemical etching;
e) subjecting the aluminium film to a second anodising step, wherein the conditions employed in this step are similar to or substantially the same as the conditions as used in the first anodising step.

According to a fifth aspect of the invention there is provided a nanoporous membrane structure, produced according to the above method.

According to a sixth aspect of the present invention there is provided a method of preparing a nanoporous membrane structure coated with viable cells comprising the steps of contacting a nanoporous membrane structure with a suspension of cells for a period of time sufficient to deposit cells onto the membrane.

According to a seventh aspect of the invention there is provided a method for preparing a homogenous population of cells, said method comprising the steps of:
a) manufacturing a nanoporous membrane structure;
b) growing cells on the nanoporous membrane structure; and
c) harvesting the cells from the membrane.

According to a eighth aspect of the invention there is provided a method of treating a patient in need of tissue damage repair, said method comprising the steps of:
a) manufacturing a nanoporous membrane structure;
b) growing cells on the nanoporous membrane structure; and
c) applying cell coated nanoporous membrane structure to a wound.

According to a ninth aspect of the invention, there is provided a method of treating a patient in need of tissue damage repair and/or cosmetic enhancement, said method comprising the steps of:
a) manufacturing a nanoporous membrane structure; and
b) applying the nanoporous membrane structure directly to the site of tissue damage.

According to an tenth aspect of the invention, there is provided a method of treating a patient in need of tissue damage repair and/or cosmetic enhancement, said method comprising the steps of:
a) manufacturing a nanoporous membrane structure;
b) contacting the nanoporous membrane structure with a solution of cultured cells; and
c) applying the nanoporous membrane structure coated with cells to the site of tissue damage.

Other aspects and advantages of the invention will become apparent to those skilled in the art from a review of the ensuing description, which proceeds with reference to the following illustrative drawings.

### BRIEF DESCRIPTION OF THE DRAWINGS

FIG. 1 Digital photograph of experimental setup of anodization process

FIG. 2 Schematic drawing of the fabrication procedure of AAO thin films:—(a) formation of the porous alumina layer after the first anodization; (b) removal of the anodic alumina layer by chemical etching; (c) formation of the ordered porous alumina layer after the second anodization; (d) chemical etching to widen the nanopores; (e) removal of protective-layer coating and addition of polymethylmethacrylate to support the film; (f) removal of the aluminium substrate; (g) chemical etching to remove barrier layer oxide; (h) removal of polymer support to afford nanoporous AAO.

FIG. 3 Schematic drawing of experimental setup A:—Nanoporous AAO was submersed in skin cell suspension, and part of membrane was removed for imaging after 1 and 24 hours. The membrane used was prepared from anodization in oxalic acid at 60 V

FIG. 4 Schematic drawing of experimental setup B:—Nanoporous AAO floated on surface of skin cell suspension, and part of membrane was removed for imaging after 1 and 24 hours. The membrane used was prepared from anodization in oxalic acid at 30 V.

FIG. 5 Digital photograph of the experimental setup of the proliferation assay, ‘P60’ denotes an AAO membrane prepared from anodization in phosphoric acid at 60V; ‘O60’ for anodization in oxalic acid at 60V; ‘O30’ for anodization in oxalic acid at 30V and ‘S24’ denotes an AAO membrane prepared from anodization in sulfuric acid at 24V. This photograph was taken after the ‘day 1’ reading.

FIG. 6 Digital photograph of aluminium strips at different stages of the development process. (a) aluminium (99.9%) strip prior to any preparative or oxidation processes; (b) AI strip after electrochemical polishing [EtOH/HCIO₄ (4:1); 16V, 80 secs]; (c) AI strip after 2° anodization process; and (d) anodic aluminium oxide membrane after removal of AI substrate.

FIG. 7 Digital photograph illustrating the development of superior AAO membranes using an optimised fabrication method.

FIG. 8 SEM image of nearly polished aluminium surface by electrochemical polishing in 4:1 EtOH/HCIO₄ at 16 V.

FIG. 9 SEM images of anodic aluminium oxide supported on aluminium.
(a) view of the surface after first anodization at 30 V and 5° C. for 7 h
(b) view of the surface after the second anodization for 5 hours using same conditions [performed after stripping in phosphoric acid/chromic acid solution at 60° C. for 1 hour.

FIG. 10 SEM images of the top surface of anodic aluminium oxide membranes at different magnifications: (a) to (c); and (d) shows the bottom of an anodic oxide membrane. Anodization was a two-step process conducted in oxalic acid (0.3 M) at 30 V and 5° C. Pore widening was performed in phosphoric acid (0.3 M) at 35° C. for 15 min. The remaining Al substrate was removed in saturated HgCl₂ solution.

FIG. 11 AFM images of the top surface of anodic aluminium oxide membranes. Anodization was a two-step process conducted in oxalic acid (0.3 M) at 30 V and 5° C. Pore widening was performed in phosphoric acid (0.3 M) at 35° C. for 15 min. The remaining AI substrate was removed in saturated HgCl₂ solution. (a) one micron AFM scan of membrane surface; and (b) 3-dimensional projection of AFM scan.

FIG. 12 SEM images of the bottom of anodic aluminium oxide membranes depicting the removal of the barrier layer oxide to afford nano-holes through the membrane. (a) chemical etching of barrier layer oxide in phosphoric acid (0.3 M) at 35° C., for 10 min; (b) chemical etching of barrier layer oxide in phosphoric acid (0.3 M) at 35° C. for 20 min.

FIG. 13 SEM images of the top surface of anodic aluminium oxide membranes at different magnifications. Anodization was a two step process conducted in oxalic acid (0.3 M) at 60 V and 5° C. Pore widening was performed in phosphoric acid (0.3 M) at 35° C. for 15 min. The remaining AI substrate was removed in saturated HgCl₂ solution.

FIG. 14 AFM images of the top surface of anodic aluminium oxide membranes prepared from anodization in oxalic acid (0.3 M) at 60 V. Pore widening was performed in phosphoric acid (0.3 M) at 35° C. for 15 min. The remaining AI substrate was removed in saturated HgCl₂ solution. (a) 2.5 μm AFM scan of membrane surface; and (b) 1.0 μm scan of membrane surface.

FIG. 15 SEM images and AFM projection of AAO membranes at different magnifications. Anodization was a two-step process conducted in sulfuric acid (0.3 M) at 24 V and 5° C. Pore widening was performed in phosphoric acid (0.3 M) at 35° C. for 15 min. The remaining AI substrate was removed in saturated HgCl₂ solution. (a) & (b) and (e)—SEM images of the top surface of the AAO membrane; (c) 3-dimensional projection of AFM scan of top surface of AAO membrane; (d) SEM image of the bottom surface of the AAO membrane; and (f) bottom of an anodic aluminium oxide membrane.

FIG. 16 SEM and AFM images of anodic aluminium oxide membranes at different magnifications. Anodization was a two step process conducted in phosphoric acid (2.5 M) at 60 V and 5° C. The remaining aluminium substrate was removed in saturated HgCl₂ solution. (a) & (b) (e) and (f) SEM images of the top surface of the AAO membrane; (c) AFM image of the top surface of the AAO membrane (5 μm); and (d) 3-dimensional projection of AFM scan of top surface of AAO membrane (2.5 μm).

FIG. 17 SEM images of the top surface of porous aluminium films formed from anodization of aluminium in tartaric acid (0.4 M) at 200 V. (a) is a view of the surface after primary anodization for 4 min. (b), (c) and (d) are views of the surface after etching and a second anodization for 2.5 min.

FIG. 18 AFM images of human keratinocytes deposited on nanoporous anodic aluminium oxide after 1 hour of contact with skin cell suspension (using setup A) [AREA A]. AAO substrate was prepared from anodization in oxalic acid (0.3 M) at 60 V. Samples were prepared for imaging by removing part of the membrane from the skin culture and washing the surface with fresh media. (a) 10 μm scan showing the coverage of cells on the AAO membrane. Image (c) is a magnification of the top right hand corner of image (b). (d) 2.2 μm scan of skin cells deposited on anodic porous alumina.

FIG. 19 AFM images of human keratinocytes deposited on nanoporous anodic aluminium oxide after 1 hour of contact with skin cell suspension (using setup A) [AREA B]. AAO substrate was prepared from anodization in oxalic acid (0.3 M) at 60 V. Samples were prepared for imaging by removing part of the membrane from the skin culture and washing the surface with fresh media. Image (b) is a magnification of the top left hand corner of image (a).

FIG. 20 AFM images of human keratinocytes deposited on nanoporous aluminium oxide after 24 hour contact with skin cell suspension (using setup A). AAO substrate was prepared from anodization in oxalic acid (0.3 M) at 60 V. Samples were prepared for imaging by removing part of the membrane from the skin culture and washing the surface with fresh media. (a) & (b) AFM images of different magnification at Area A; and (c) & (d) AFM images of different magnification at Area B.

FIG. 21 AFM images of human keratinocytes deposited on nanoporous aluminium oxide after 1 hour of contact with skin cell suspension (using setup B). AAO substrate was prepared from anodization in oxalic acid at 30 V. Samples were prepared for imaging by removing part of the membrane from the skin culture and washing it with fresh media. (a) & (b) AFM images of different magnification at Area A; and (c) & (d) AFM images of different magnification at Area B.

FIG. 22 AFM images of human keratinocytes on deposited on nanoporous aluminium oxide after 24 hour contact with skin cell suspension (using setup B). AAO substrate was prepared from anodization in oxalic acid at 30 V. Samples were prepared for imaging by removing part of the membrane from the skin culture and washing it with fresh media. (a), (b) & (c) AFM images of different magnification at Area A; and (d) AFM image of cells deposited on AAO at Area B.

FIG. 23 Screen captures from in-situ real-time light microscopy of skin cell behaviour on nanoporous anodic aluminium oxide over a period of 24 hours. The underlying AAO membrane (not visible in these images) was prepared from anodization in phosphoric acid at 60V. Cell behaviour was recorded (using a camcorder attached to the microscope) and complied into a movie that can be accessed electronically in Appendix A (mov format). The series of screen captures illustrate the adhesion and proliferation of human keratinocytes on nanoporous anodic aluminium oxide. (a) screen capture at time-0 showing round (typsinised) skin cells not yet anchored to the AAO membrane; (b) screen capture of the sample after 4 hours showing trypsinised cells return to their normal morphology and are attaching to the surface; (c) screen capture of sample after 12 hours, highlighting cell proliferation; (d) enlargement of specified area in image (c); and (e) screen capture of sample after 24 hours. Imaging was performed at 20× magnification.

FIG. 24 Proliferation assay results. The quantity of formazan product as measured by the absorbance at 492 nm is directly proportional to the number of living cells in culture. As evident in the graph, there is a noticeable difference in the proliferation on the membrane with pore separation of 160 nm (i.e. that prepared in oxalic acid at 60V) when compared to the other membranes.

FIG. 25 Proliferation assay results. The quantity of formazan product as measured by the absorbance at 492 nm is directly proportional to the number of living cells in culture. As evident in the graph, there is a noticeable difference in the proliferation on the membrane with pore separation of 150 nm (i.e. that prepared in oxalic acid at 60V) when compared to the other membranes.

### DETAILED DISCLOSURE OF THE INVENTION

### General

Those skilled in the art will appreciate that the. invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations of any two or more of the steps or features.

The present invention is not to be limited in scope by the specific embodiments described herein, which are intended for the purpose of exemplification only. Functionally equivalent products, compositions and where appropriate methods are clearly within the scope of the invention as described herein.

Throughout this specification and the claims that follow, unless the context requires otherwise, the word “comprise”, or variations such as “comprises” or “comprising”, will be understood to imply the inclusion of a stated integer or group of integers but not the exclusion of any other integer or group of integers.

### DESCRIPTION OF PREFERRED EMBODIMENTS

Anodic aluminium oxide (AAO) is a highly regular and porous structure, which is produced from the anodic oxidation of aluminium in acidic electrolytes. Unlike other tissue engineering scaffolds, AAO is a totally inert, inorganic membrane. Aluminium oxide has long been appreciated for its biocompatibility in clinical applications and thus overcomes the concerns of unfavourable responses to polymeric scaffolds. In addition, AAO is a 2-dimensional substrate that does not aim to entrap cells in an attempt to organise them into 3-dimensions. It provides a substrate to which tissue or more particularly cells can attach, interact and organise without overtly forcing cell arrangement. This is desirable, as it allows easy removal of cells from the substrate when needed, since they are not trapped deep within a 3-dimensional construct.

Under specific anodizing parameters, the self-organised oxide growth generates a densely packed, hexagonal array of uniform-size pores, which are almost perfectly aligned perpendicular to the surface of the AAO film. This resulting template is characterised by anodization at high voltages and forms in oxalic, sulphuric, phosphoric, tartaric and malonic acid solutions with inter-pore distances ranging from 50-420 nm and more preferably from 10-500 nm.

According to a first aspect the invention provides a nanoporous membrane structure characterised in that it has a hexagonal array of tubes with a substantially uniform inter-pore distance between the tubes wherein the distance is within the range from 10-420 nm and more preferably between the range from 10-500 nm, and wherein the tubes may have a depth of up to 500 μm. The pore density is usually in the range from 10⁹ to 10¹² pores/cm². Preferably the tubes are aligned substantially perpendicular to the surface of the membrane.

As used herein “tubes with a substantially uniform inter-pore distance” refers to there being a relatively consistent inter-pore distance between the tubes forming the membrane. Preferably the inter-pore distance is within the range from 50 to 350 nm, more preferably it is within the range of 55 to 300 nm, 60 to 270 nm, 60 to 240 nm, 65 to 200 nm, 70 to 150 nm, and 75 to 125 nm. By changing the electrolyte and voltage parameters of the system, ordered pore structures with inter-pore distances of approximately 50, 60, 100, 150, 420 and 500 nm are achievable.

The pore diameter and periodicity of the AAO templates may be controlled, thus allowing for the ‘nano-engineering’ of the pore geometry by changing the macroscopic parameters, such as the anodization time and voltage, the anodizing electrolyte and/or the time of post chemical etching.

In an alternate form of the present invention the nanoporous membrane structure the inter-pore distance will gradually decrease from the periphery of the membrane to the centre of the membrane. In yet another alternate form of the invention the inter-pore distance will gradually increase from the periphery of the membrane to the centre of the membrane. Further the membrane may comprise a hexagonal array of tubes with a substantially uniform-inter-pore distance towards the centre of the membrane but a different uniform-inter-pore distance towards periphery of the membrane

According to the second aspect of the invention there is provided a method for preparing a nanoporous membrane structure. Preferably the method comprises the steps of:
a) preparing an aluminium film;
b) subjecting the aluminium film to a first anodising step;
c) subjecting the aluminium film to wet chemical etching;
d) subjecting the aluminium film to a second anodising step, wherein the conditions employed in this step are similar to or substantially the same as the conditions as used in the first anodising step.

According to a preferred embodiment, the invention provides a method comprising the steps of
(a) preparing an aluminium film;
(b) subjecting the aluminium film to a first anodising step;
(c) subjecting the aluminium film to wet chemical etching;
(d) subjecting the aluminium film to a second anodising step, wherein the conditions employed in this step are similar to or substantially the same as the conditions as used in the first anodising step;
(e) contacting the aluminium film form step (d) with a material capable of supporting the film;
(f) suspending the aluminium film in a solution which completely removes the unprotected alumina substrate; and
(g) removing the layer of oxide to produce nano-channels throughout the alumina film

According to the method of the invention the first anodization step is carried out using a constant voltage ranging from between about 15 and 200 volts. Preferably the voltage selected is between 20 and 80 volts. Even more preferably it is about 25, 30, 35, 40, 45, 50, 55, 60, 65, 70 or 75 volts. For example the voltage might be 30 volts where an oxalic acid solution is used in this step of the method. In another example, the voltage might be 200 volts when tartaric acid solution is used in this step of the method.

The acid used in the first anodization step will be an aqueous solution containing a di- or triprotic acid. Sulphuric acid, oxalic acid, phosphoric acid, chromic acid, tartaric acid and malonic acid can be used as the acid.

The anodic oxidation for production of the membranes to be employed according to the invention is usually carried out at a low temperature, for example 0 to 5° C., and preferably using sulphuric acid or oxalic acid as the electrolyte, because thick, compact, and hard porous films are obtainable in this way. However, when other acids are used, such as tartaric acid or malonic acid, the anodic oxidation step may be carried out at room temperature.

The period of time over which the first anodization step is carried out will depend on the acid employed, its concentration, the voltage and the temperature. Typically, however, the step will be carried out for at least 5 hours. More preferably the step is carried out for 5.5, 6, 6.5, 7, 7.5, 8, 8.5, 9 or at least 9.5 hours.

When the first anodization step is carried out using tartaric. acid or malonic acid, the step may be carried out from between 1 minute to about 10 minutes. Preferably, the step is carried out for 1.5, 2.0, 2.5, 3.0, 3.5, 4.0, 4.5, 5.0, 6, 7, 8, 9 or 10 minutes.

Where the method of the invention includes a wet chemical etching step the anodized section of the strip was then exposed to an acid solution for a period of time to remove the alumina layer formed on the unprotected side of the sheet. For example, a mixture of phosphoric acid and chromic acid can be used at 60° C. for 1 hour.

According to the method of the invention the second anodization was performed for a suitable time period under the same conditions as the first anodization. So for example the second anodization step might be carried out using 0.3 M oxalic acid, with a voltage of 30 V, and a temperature of 5° C. for a time of 5 hours. In another example, the second anodization step might be carried out using 0.4 M tartaric acid, with a voltage of 200 V, at room temperature for 2.5 minutes. Using such a set of parameters the inventors have been able to prepare nanoporous layer of alumina which serves as the final membrane with very well defined pore size architecture.

To widen the nanopores in the membrane, the membrane can be subjected to further chemical etching, by applying a suitable acid to the membrane for a suitable amount of time. For example the acid could be phosphoric acid (0.3 M) and that might be applied for 10 to 20 but more preferably 15 minutes.

According to the method of the invention, once the membrane has been through the second anodization step the membrane is coated with a material to support it. Preferably that support is a liquid acrylic such as to polymethyl methacrylate, PMMA, like for example “Acrifix 92, now known as Acrifix 192”. The film serves to support the AAO membrane during removal of the aluminium substrate.

To remove the unprotected aluminium substrate, the membrane is then contacted with a solution of mercuric chloride.

To remove the barrier layer of oxide and produce nano-channels throughout the alumina film, the supported AAO was further etched in an acid solution for a suitable amount of time at room temperature. For example the acid might be phosphoric acid (0.3 M), applied for 20 minutes at room temperature.

In a highly preferred embodiment the present invention provides a method for preparing a nanoporous membrane structure suitable for use in treating damaged tissue, comprising the steps of:
a) polishing an aluminium film;
b) applying a layer of polymer film to one side of the aluminium film;
c) performing a first anodising step to the aluminium film;
d) wet chemical etching of the aluminium film;
e) performing a second anodising step to the aluminium film;
f) removing the polymer film;
g) applying an acrylic film to support the aluminium film;
h) removing unwanted layers of alumina formed during the anodization processes;
i) suspending the aluminium film in a solution which completely removes the unprotected aluminium substrate; and
j) removing the formed layer of oxide to produce nanochannels throughout the alumina film.

In an even more preferred form the invention includes the step of chemically etching the alumina film to widen nanopores between the step (e) and (f) in the above method.

According to a third aspect of the invention there is provided a nanoporous membrane structure, produced according to the above method.

According to a fourth aspect of the invention, there is provided a method for preparing a nanoporous membrane, comprising the steps of:
a) preparing an aluminium film;
b) applying a protective layer to one side of the aluminium film;
c) subjecting the aluminium film to a first anodising step;
d) subjecting the aluminium film to wet chemical etching;
e) subjecting the aluminium film to a second anodising step, wherein the conditions employed in this step are similar to or substantially the same as the conditions as used in the first anodising step.

Preferably, the protective layer is a polymer, such as ethyl acetate and butyl acetate. Other protective layers known to those skilled in the art may be used, such as silicon. In a highly preferred embodiment, the protective layer is removed after the second anodising step has been completed.

According to a fifth aspect of the invention there is provided a nanoporous membrane structure, produced according to the above method.

A person skilled in the art will realise that a highly ordered nanopore membrane as described herein will have a wide range of potential uses. One such use it which the membrane may be applied is as a “blueprint” in tissue engineering. In this respect blue print has been found in our studies to positively influence cell behaviour and enhance proliferation, differentiation, cell adhesion, interaction and organisation.

Through the use of the present invention cell proliferation and differentiation can be manipulated in a manner that facilitates tissue engineering and wound healing. For example successful optimisation and enhancement of keratinocyte behaviour will reduce the time to heal and enhance wound repair and scar quality. In addition, skin cells which readily respond to the AAO substrate, may potentially develop a fast structured growth mechanism that can be carried over to a wound bed in the form of a cell ‘band-aid’. Faster coverage of a wound through enhanced proliferation would be a significant advancement in the treatment of wounds.

Accordingly, in an embodiment of the invention the nanoporous membranes may be prepared as a scaffold upon which tissue regeneration may take place. Hence the nanoporous membranes may be provided as a dressing or as a bandaid or closure for a wound.

It will be appreciated that where the invention is prepared as a tissue dressing the membrane may be impregnated with therapeutic compounds or pharmaceutically desirably compounds to aid in the regeneration of the tissue. For example the membrane may be impregnated with proteins such as cytokines, anti-infective agents such as penicillins, cephalosporins, aminoglycosides, miscellaneous agents such as aztreonam, bacitracin, ciprofloxacin, clindamycin, chloramphenicol; cotrimoxazole, fusidic acid, imipenem, metronidazole, teicoplanin, and vancomycin, antifungals, antivirals, antineoplastic agents, alkylating agents, antibiotics, antimetabolites, antifolates, immunosuppressant agents, anti-angiogenesis agents, anti-inflammatory or suppressive factors. Preferably such agents are loaded into the membrane by diffusion, coating, spraying, or through an ion transfer process.

According to a sixth aspect of the present invention there is provided a method of preparing a nanoporous membrane structure coated with viable cells comprising the steps of contacting a nanoporous membrane structure with a suspension of cells for a period of time sufficient to deposit cells onto the membrane.

According to a seventh aspect of the invention there is provided there is provided a method for preparing a homogenous population of cells, said method comprising the steps of:
a) manufacturing a hanoporous membrane structure;
b) growing cells on the nanoporous membrane structure; and
c) harvesting the cells from the membrane.

Preferably, the method prepares a homogenous population of ordered cells.

Cells produced according to the above method may be harvested by either physical or chemical disruption means. Physical disruption might include, for example, scraping the cells off the membrane. Chemical disruption might include, for example, digestion with enzymes such as trypsin, dispase, collagenase, trypsin-edta, thermolysin, pronase, hyaluronidase, elastase, papain and pancreatin. Non-enzymatic solutions for the dissociation of tissue can also be used.

Preferably, disruption of the cells is achieved by placing the membrane on which the cells are growing in a pre-warmed enzyme solution for example a trypsin solution, however, any other enzyme such as dispase, collagenase, trypsin-edta, thermolysin, pronase, hyaluronidase, pancreatin, elastase and papain that cause cells to become detached from other cells or from solid surfaces. When the enzyme used is trypsin the enzyme solution used in the method is preferably calcium and magnesium free. One such solution is preferably calcium and magnesium ion free phosphate buffered saline.

The amount of trypsin that might be used in the method is preferably between about 5 and 0.1% trypsin per volume of solution. The desirable trypsin concentration of the solution is about 2.5 to 0.25%, with about 0.5% trypsin being most preferred.

The time period over which the cell population is subjected to the trypsin solution may vary depending on the size of the cellular mass. Preferably, the cells are placed in the presence of the trypsin solution for sufficient time to weaken the cohesive bonding between the cells. For example, the cells might be placed in trypsin for a 5 to 60 minute period.

After the tissue sample has been immersed in the trypsin solution for an appropriate amount of time, the sample is removed from the trypsin and washed with nutrient solution. Washing the tissue sample may involve either partial or complete immersion of the treated sample in the nutrient solution. Alternatively, and more preferably, the wash solution is dripped on the tissue sample in sufficient volume to remove and or significantly dilute any excess trypsin solution from the surface of the sample. Preferably any dilution that might occur would lead to less than 0.05% trypsin in the nutrient solution.

The nutrient solution used in the method should be capable of significantly reducing and more preferably removing the effect of the trypsin either by dilution or neutralisation. The nutrient solution used in the method will also preferably have the characteristics of being (i) free of at least xenogenic serum, (ii) capable of maintaining the viability of the cells until applied to a patient, and (iii) suitable for direct application to a region on a patient undergoing tissue grafting. The solution may be anything from a basic salt solution to a more complex nutrient solution. Preferably, the nutrient solution is free of all serum but contains various salts that resemble the substances found in body fluids; this type of solution is often called physiological saline. Phosphate or other non-toxic substances may also buffer the solution in order to maintain the pH at approximately physiological levels. A suitable nutrient solution that is particularly preferred is Hartmann's solution.

According to a eighth aspect of the invention there is provided a method of treating a patient in need of tissue damage repair, said method comprising the steps of:
a) manufacturing a nanoporous membrane structure;
b) growing cells on the nanoporous membrane structure; and
c) applying cell coated nanoporous membrane structure to a wound.

According to a ninth aspect of the invention, there is provided a method of treating a patient in need of tissue damage repair, said method comprising the steps of:
a) manufacturing a nanoporous membrane structure; and
b) applying the nanoporous membrane structure directly to the site of tissue damage.

According to an tenth aspect of the invention, there is provided a method of treating a patient in need of tissue damage repair, said method comprising the steps of:
a) manufacturing a nanoporous membrane structure;
b) contacting the nanoporous membrane structure with a solution of cultured cells; and
c) applying the nanoporous membrane structure coated with cells to the site of tissue damage.

### BEST MODE(S) FOR CARRYING OUT THE INVENTION

These and other advantages will become apparent from the examples set forth below. Such examples are provided only for exemplification of the invention and are not to be considered as limiting the invention.

### EXAMPLES

### Fabrication of Anodic Aluminium Oxide Thin Films

### Fabrication of AAO Films Using Oxalic, Sulphuric or Phosphoric Acid

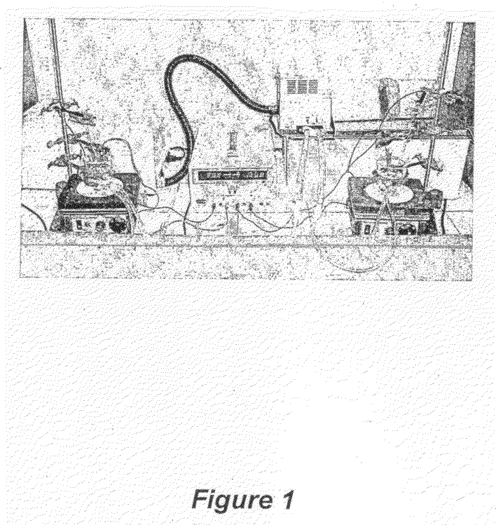

Anodic aluminium oxide (AAO) thin films were prepared using the experimental setup shown in FIG. 1. More specifically, flat nanoporous alumina films were made from an aluminium sheet using a two-step anodization process. First, high purity (99%) aluminium strips (˜6×1 cm or 5×1.5 cm) were annealed under argon in a quartz tube at 500° C. for 5 hours. This was performed to recrystallise the samples and release all mechanical stress from the structure. The substrates were subsequently cleaned by ultra-sonication in acetone for 20 minutes.

A 2×1.5 cm or 4×1.5 cm section of the aluminium strip was electrochemically polished in a 4:1 v/v solution of ethanol/perchloric acid to a mirror finish. The strip was made the anode of an electrochemical cell by vertically suspending it opposite a platinum electrode (cathode) in the stirred electrolyte. A constant voltage of 16 V was supplied using a regulated DC power supply (Good Will Instruments; Model GPC:3030D), between the electrodes for 80 seconds at room temperature. The aluminium piece was thoroughly washed with ethanol and then acetone before a thin layer of polymer was applied to one side of the substrate. The polymer used in this work was ethyl acetate and butyl acetate (i.e. fingernail polish).

The polymer-coated aluminium strip acted as the anode of an electrochemical cell and was suspended parallel to a Pt electrode. The temperature of the electrolyte solution was controlled by pumping water from a water bath to a jacketed cell vessel. Only the polished section of the aluminium strip was immersed in the electrolyte for anodization.

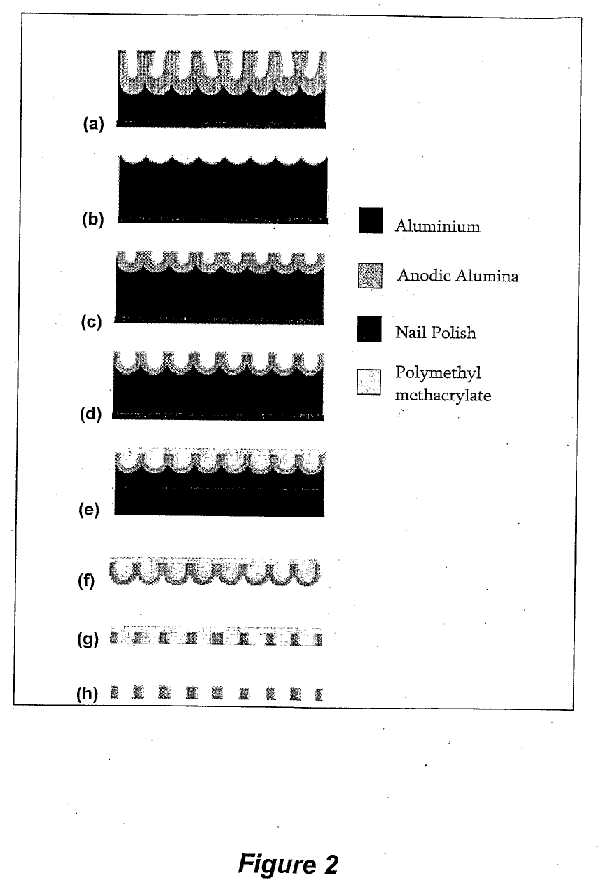

The process of developing nanoporous AAO is schematically shown in FIG. 2. Using the setup in FIG. 1, the aluminium sheet was anodized at a constant dc voltage of 30 V in oxalic acid aqueous solution (0.3 M) at 5° C. for 7 to 8 hours (FIG. 2a). The anodized section of the strip was then exposed to a mixture of phosphoric acid and chromic acid (35 mL of 85% H₃PO₄+10 g CrO₃; made to 500 mL with MilliQ water) at 60° C. for 1 hour. The wet chemical etching technique was performed to remove the alumina layer formed on the unprotected side of the sheet. The anodization/removal step leaves a uniform concave nano-array in the aluminium substrate that is crucial for achieving the narrow pore size distribution during the subsequent anodization step (FIG. 2b).

With one side of the aluminium substrate still protected by the polymer film, a second anodization was performed for 5 hours under the exact same conditions as the first [oxalic acid (0.3 M), 30 V, 5° C.]. This resulted in a nanoporous layer of alumina which serves as the final membrane with very well defined pore size architecture (FIG. 2c). The nanopores were widened by chemical etching in phosphoric acid (0.3 M) at 35° C. for 15 minutes (FIG. 2d).

The protective polymer film was then completely removed by acetone and a cotton swab. Following this step, a thin and even coat of “Acrifix 92” (Acrifix 192), a liquid acrylic (polymethyl methacrylate, PMMA) product was applied to the anodized side of the aluminium strip (FIG. 2e). The acrylic film served to support the AAO membrane in the removal of the aluminium substrate. Sodium hydroxide (3 M) and a cotton swab were used to completely remove an unwanted layer of alumina that had formed underneath the ‘protective’ polymer coating during the anodization processes. The entire piece was then thoroughly rinsed in milli-Q water.

The strip was suspended in a saturated solution of mercuric chloride (stirred) to completely remove the unprotected aluminium substrate (˜30 minutes) (FIG. 2f). To remove the barrier layer of oxide and produce nano-channels throughout the alumina film, the supported AAO was further etched in phosphoric acid (0.3 M) for between 10 minutes to 20 minutes at room temperature (FIG. 2g). Complete dissolution of the acrylic support in acetone afforded the AAO membrane as a clear thin film (FIG. 2h).

To fabricate AAO membranes with a range of pore sizes, different anodizing conditions were investigated. The overall procedure was the same for each preparation, with the exception of the parameters given in Table 1. In addition, no pore widening procedure was performed on AAO membranes prepared in phosphoric acid at 60 V.

TABLE 1

| | | Time of 1° | | Time of 2° |
|---|---|---|---|---|
| Anodizing Acid | Voltage | Anodization | | Anodization |
| Oxalic acid (0.3 M) | 60 V | 5 | hours | 3 hours |
| Sulfuric acid (0.3 M) | 24 V | 5 | hours | 3 hours |
| Phosphoric acid (2.5 M) | 60 V | 7 or 8 | hours | 5 hours |

### Cleaning/Sterilisation of Alumina Membranes

The alumina membranes were boiled in 30% hydrogen peroxide for 15 minutes to clean and sterilise the surface. Furthermore, the films were boiled in milli-Q water for 15 minutes, and then air-dried and stored in an airtight container until needed.

### Fabrication of AAO Films Using Tartaric Acid or Malonic Acid

Anodic aluminium oxide thin films with a pore size of between 200 nm and up to 500 nm were prepared using an experimental setup similar to that shown in FIG. 1. More specifically, flat nanoporous alumina films were made from an aluminium sheet using a two-step anodization process. First, high purity (99%) aluminium strips (˜6×1 cm or 5×1.5 cm) were annealed under argon in a quartz tube at 500° C. for 5 hours. This was performed to recrystallise the samples and release all mechanical stress from the structure. The substrates were subsequently cleaned by ultra-sonication in acetone for 20 minutes.

The electrochemically polishing step as described above was not carried out on the 4×1.5 cm section of the aluminium strip used in this method. However, the effect of the electrochemically polishing step will be examined in more detail at a later date.

The aluminium strip acted as the anode of an electrochemical cell and was suspended parallel to a Pt electrode. The temperature of the electrolyte solution was controlled by pumping water from a water bath to a jacketed cell vessel.

Using the similar setup in FIG. 1, the aluminium sheet was anodized at a constant dc voltage of 200 V in tartaric acid aqueous solution (0.4 M) at room temperature for 4 minutes, whilst stirring the solution. The anodized section of the strip was then exposed to a mixture of phosphoric acid and chromic acid (35 mL of 85% H₃PO₄+10 g CrO₃; made to 500 mL with MilliQ water) at 60° C. for 1 hour. The wet chemical etching technique was performed to remove the alumina layer formed on the sheet. The anodization/removal step leaves a uniform concave nano-array in the aluminium substrate that is crucial for achieving the narrow pore size distribution during the subsequent anodization step.

A second anodization was performed for 2.5 minutes under the exact same conditions as the first [tartaric acid (0.4 M), 200 V, room temperature]. The film was subjected to a “pore-widening”, chemical etching step, as described above for the other membranes. However, “pore-widening” may also be preformed in a 20 g/L CrO₃/35 mL/L H₃PO₄ etching solution at 60° C. for times of up to 30 minutes.

This resulted in a nanoporous layer of alumina which serves as the final membrane with very well defined pore size architecture. No further surface manipulation was performed before analysis by SEM.

A similar method is intended to apply using malonic acid, using a voltage of 90 V.

### Surface Characterisation of Anodic Aluminium Oxide Thin Films

### Scanning Electron Microscopy

The porous structure of the alumina membranes was imaged using scanning electron microscopy (SEM) on a Philips XL 30 SEM instrument. In addition, the important stages of the development process were characterised, which involved imaging the aluminium substrate/AAO membrane after;
the electrochemical polishing process;
the anodization processes;
the chemical etching processes.

Samples were prepared for SEM by mounting them on aluminium ‘stubs’ covered in carbon tape. The alumina membranes, like other non-conductive surfaces, were sputter coated with gold to minimise the negative charge accumulation on the sample surface. The sputter coater was set at a current of 40 mA and pressure of 2.5-3×10⁻¹ torr for 1 minute to deposit a thin gold layer. Subsequently, secondary electron images were recorded at voltages of 10-15 kV at various magnifications.

### Atomic Force Microscopy

Additional investigation of the surface topography was performed by Atomic Force Microscopy (AFM). Samples were prepared by setting a small piece of the AAO thin film (˜5×5 mm) to a ‘steel stub’ that had been covered with double sided 3M Scotch Tape. AFM scans were performed using micro-fabricated silicon cantilevers with silicon nitride sharpened tips in contact mode. Contact-mode was chosen over tapping-mode since this method significantly improves lateral resolution on porous surfaces and thin films. The AFM images were obtained under ambient laboratory conditions. Image analysis and parameter calculations were performed using AFM images recorded on a Digital Nanoscope E instrument (Digital Instruments/Veeco). The images were recorded and ‘flattened’ to correct for the inherent curvature of the AFM scanner using ‘Nanoscope® III Digital Instruments Software’ version 5.12r³⊚2001.

### Characterisation of AAO Thin Films

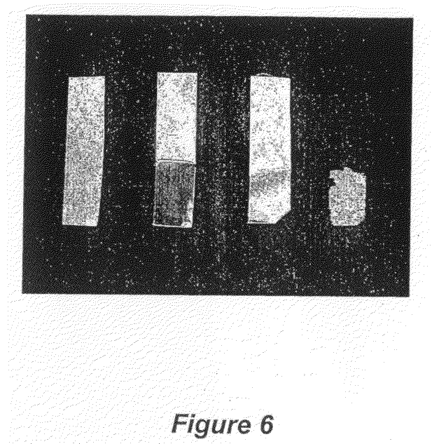

As discussed above, the surface topography of AAO membranes was characterised by Scanning Electron Microscopy (SEM) and Atomic Force Microscopy (AFM). However, it is interesting to note the visual characteristics of the aluminium substrate at different stages of the fabrication process. It is clear from FIG. 6 that structural changes occurring at the nanoscale during AAO formation have a direct effect on the appearance of the aluminium substrate.

FIG. 6a depicts an aluminium strip prior to any preparative or oxidative processes, while FIG. 6b shows the aluminium substrate after electrochemical polishing. The polishing process effectively removes the layer of naturally formed aluminium oxide to afford a smooth, stress-free surface for anodization. The shiny, mirrored finish that is observable in FIG. 6b is consistent with this process. Anodization of the aluminium substrate essentially replaces the aluminium oxide in an ordered configuration and FIG. 6c depicts the matte alumina surface. Lastly, FIG. 6d illustrates the final unsupported AAO membrane as a clear and transparent thin film that is of the same size as the anodized section of the aluminium strip. These visual cues were evident in each preparation, regardless of the anodizing conditions used.

### Optimisation of Technique to Develop Superior AAO Membranes

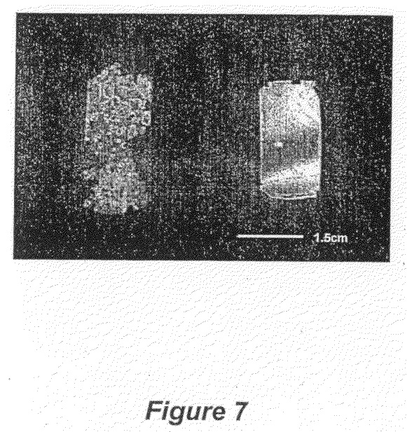

Optimisation of the preparative methods of AAO thin films has resulted in the fabrication of superior membranes as illustrated in FIG. 7. The AAO membrane shown in FIG. 7 was prepared using known procedures of two-step anodization technique for the fabrication of porous anodic alumina. It is clear from the image that the resulting membranes were of very poor quality, having a flaky and delicate nature that would not be beneficial for tissue engineering applications. The main reason for the inferior quality is thought to be due to the penetration of the mercuric chloride solution through the alumina films during the removal of the aluminium substrate. This problem was overcome by coating one side of the aluminium strip with nail polish (prior to anodization) to form a protective polymer coat and essentially block oxidation of that side. Once the polymer coating was removed, it allowed the mercuric chloride solution to have direct contact with the aluminium substrate and permit a clean and fast removal without damaging the overlaying membrane. This simple, yet high effective modification of the technique afforded superior quality membranes as depicted on the right in FIG. 7.

It was also found to be less difficult to recover a single large sheet of AAO membrane if it was supported during the removal of the aluminium substrate. As a result, a thin coat of polymethyl methacrylate (PMMA) was applied to the anodised side of the aluminium strip before it was immersed in the mercuric chloride solution. After complete removal of the substrate, dissolution of the polymer support provided a single sheet of AAO which was the same size as the anodized region. Overall, optimisation of the two-step anodization technique resulted in the fabrication of superior alumina membranes to those previously reported.

### Preparation of the Aluminium Substrate for Anodization

There are two important processes required for the formation of highly regular nano-structured pores in anodic alumina. Sufficient annealing is necessary to recrystallise and remove all mechanical stresses from the aluminium substrate to provide homogeneous conditions for oxide growth. In addition, the aluminium surface is commonly electropolished to remove the naturally formed aluminium oxide on the surface.

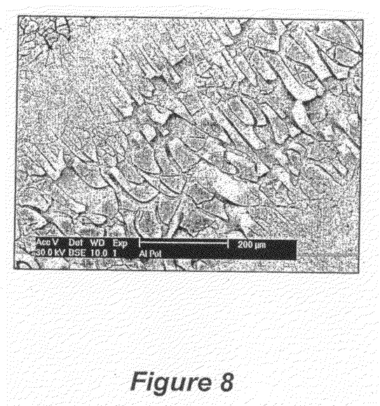

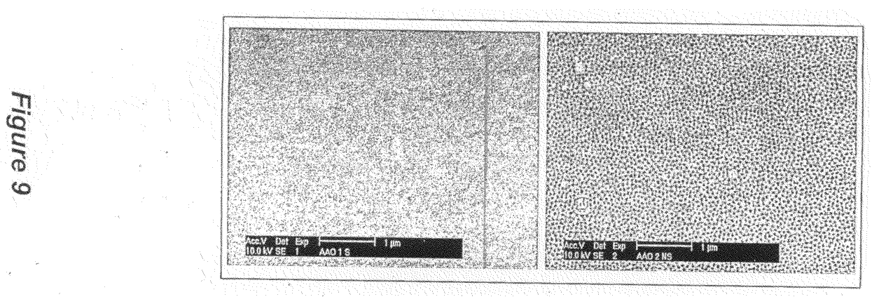

FIG. 8 is an SEM image of a ‘nearly polished’ strip of aluminium by the electrochemical method, where it can be seen that the process has begun to remove the oxide layer by essentially peeling it from the underlying aluminium. The image also illustrates the importance of this pre-treatment step since a smooth, clean surface is required for anodization.

### The Two-Step Anodization Process and Ordered Pore Arrays

The advanced two-step anodization technique is the preferred method of preparing AAO thin films as it decreases the overall anodization time required to afford ordered and parallel nano-channels throughout the alumina layer. FIG. 9 illustrates SEM images that characterise the two-step anodization method and the development of nanoporous arrays.

FIG. 9a is an SEM image of the aluminium strip after the first anodization. It illustrates the surface of the aluminium oxide to be relatively flat with no ‘visible’ pores. However, FIG. 9b is an SEM image (of similar magnification) of the aluminium strip after the second anodization. The image clearly shows a densely packed hexagonal array of cylindrical pores of uniform size in the surface of the aluminium oxide layer. It is important to note that the regular nanoporous architecture was observed across the entire alumina film. The formation of the ideal honeycomb structure after the brief second anodization supports suggestions in the literature that the first anodization changes the surface of the aluminium substrate into an undulating landscape that is consistent with the spatial ordering of the barrier layer. Fast and structured growth is observed in the subsequent anodization since the substrate acts as an ordered template, directing pore initiation positions and therefore eliminating the need for long-term self-ordering. This allows and facilitates the growth of ordered nano-channels throughout the alumina layer, and in contrast to the primary anodization. (FIG. 9a), regular pore formation is observable in the surface of the AAO film (FIG. 9b).

### Characterisation of AAO Thin Films Prepared in Oxalic Acid at 30 V

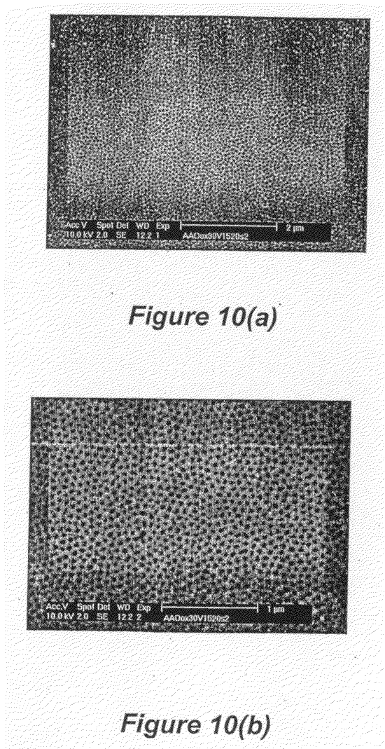

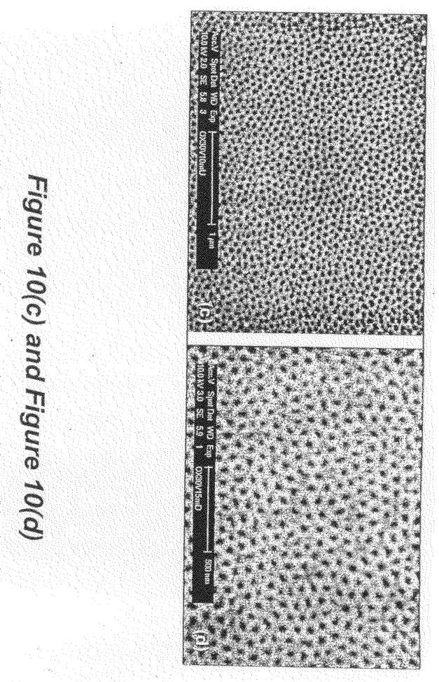

AAO membranes were successfully characterised by SEM and AFM techniques. FIG. 10 illustrates SEM images of the top surface of an AAO membrane prepared in oxalic acid at 30V.

The images illustrate the successful preparation of nanoporous anodic alumina from the optimised two-step anodization technique. It is evident from the magnified view in FIG. 10b that the pores are of uniform size and exist in an ideal hexagonal array. The surface topography is similar to honeycomb and the image shows the presence of many ordered pore domains in the surface. FIG. 10a presents a view of the AAO membrane surface to portray the existence of the highly ordered architecture across the entire surface of the thin film.

The diameter of the pore mouth and pore separation (inter-pore distance) were measured using sectional profile lines during imaging (for all the preparations). For the evaluation of the pore mouth diameter, profile marks were visually set on the upper inflexion points of the conical profile lines, and the pore separation was estimated by placing the marks in two adjacent minima of the same profile lines. The two-step anodization of aluminium in oxalic acid at 30 V produced pore mouth diameters of ˜55 nm and pore separations (or inter-pore distances) of ˜80 nm.

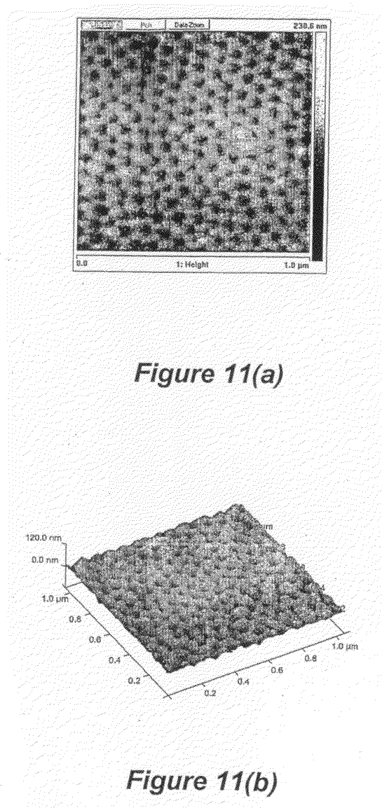

Atomic force microscopy images of the prepared membranes are given in FIG. 11. In support of the SEM characterisation, the AFM images also show a very high aspect ratio of nanopores in the surface of the AAO thin film. The 3-dimensional projection given in FIG. 11b portrays the nanoporous topography that is to be investigated in regards to skin cell behaviour. Pore geometry measurements were consistent with those reported from the SEM images.

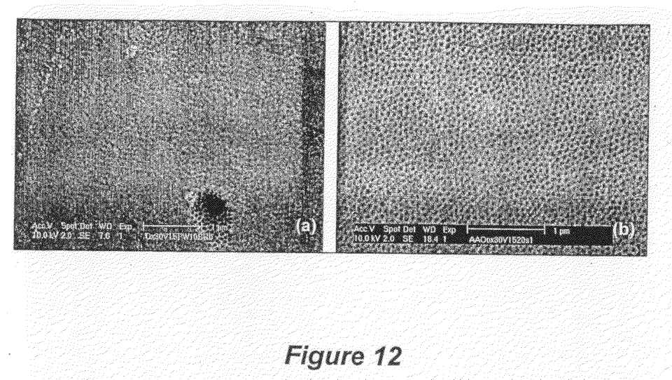

The process of removing the barrier layer oxide to afford nano-channels throughout the alumina film was also characterised by SEM. FIG. 12 presents SEM images of the bottom surface of the alumina film after exposure to phosphoric acid for differing times. It is clear from FIG. 12b that near-complete removal of the barrier layer oxide was achieved in 20 minutes to form a true, nanoporous film. The image also shows the etched surface to possess the same pore arrangement and geometry as its surface counterpart. The topography of the barrier layer oxide is shown in FIG. 12a and is characterised by hemispherical protrusions, uniform in size, which correspond to the bottom of the nanopores. This observation also indicates that a regular self-organised growth takes place at the metal/oxide interface. The membrane shown in FIG. 12a was only exposed to phosphoric acid for 10 minutes, and was obviously insufficient to completely etch the bottom of the pores (with the exception of the small defect at the bottom of the image).

### Characterisation of AAO Thin Films Prepared in Oxalic Acid at 60 V

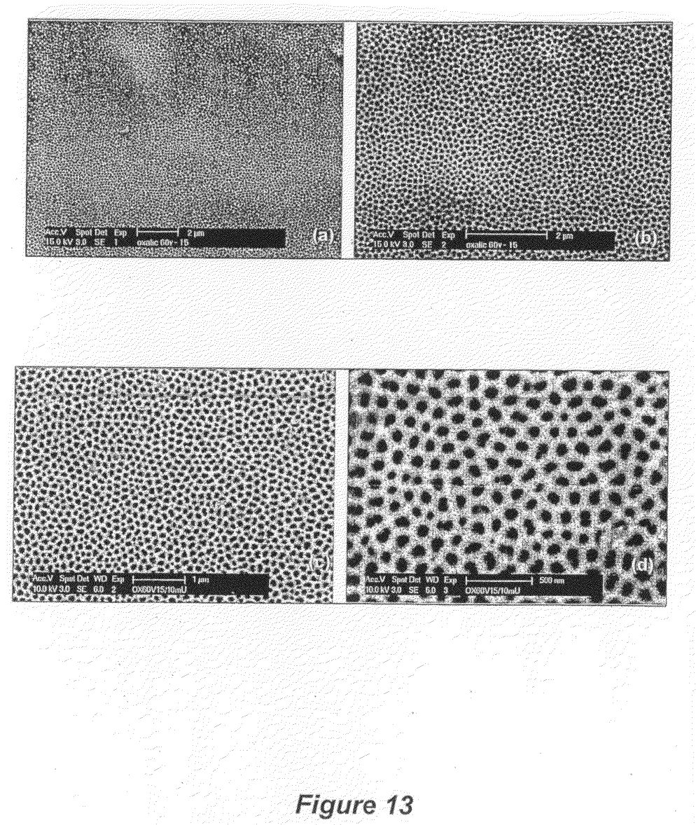

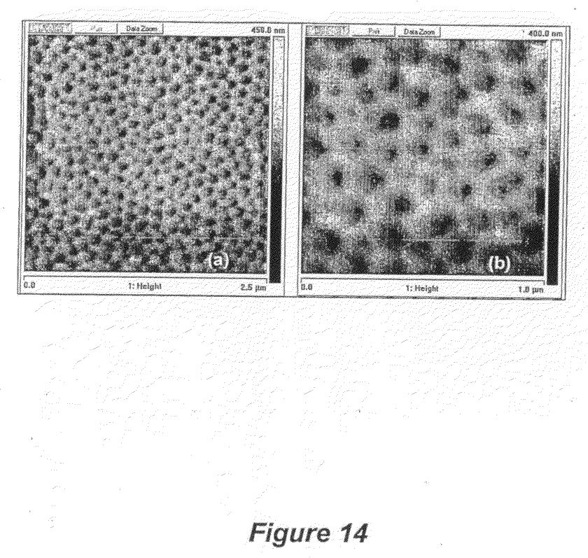

AAO membranes prepared under a higher anodizing voltage in oxalic acid were also characterised by SEM (FIG. 13) and AFM (FIG. 14). The larger view of the membrane surface given in FIG. 13a depicts the highly ordered nano-architecture to exist across the entire surface of the prepared thin film. It is clear from FIG. 13b that anodization at the higher voltage produces a characteristically different membrane to that formed at 30 V. While the majority of pores exist in ordered domains and the ideal hexagonal structure is apparent, it is also clear that some pores deviate from the regular cylindrical shape. This is more evident in the high resolution AFM scan in FIG. 14b where it seems that neighbouring pores have merged to form more of an elliptical shape. This observation is most likely due to the unstable growth of the oxide at the higher voltage, and the enhanced field-assisted dissolution of the oxide layer under the higher forming voltage.

It is also noticeable that there is a reduced degree of ordering across the membrane surface due to the irregular shaped pores. Unlike that observed in the membrane prepared at 30 V, regular hexagonal arrays of nanopores only exists in small domains and is likely due to the enhanced dissolution of oxide at the pore bottom under the higher field.

The enhanced dissolution of the oxide at the higher voltage is also a likely reason for the observed increase in pore diameter and pore separation (inter-pore distance). As detailed above, there is increased dissolution of the barrier layer oxide at the pore bottom under higher fields, as well as the change in the mechanism of oxide growth at the high voltage. Overall, the increased dissolution at the pore bottom appears to increase the pore diameter to form self-assembled cells at larger intervals. The anodization of aluminium in oxalic acid at 60 V produced porous anodic alumina with a majority of cylindrical pores having an average diameter of between about 110-120 nm and a pore separation (inter-pore distance) of between about 150-160 nm.

### Characterisation of AAO Thin Films Prepared in Sulfuric Acid at 24 V

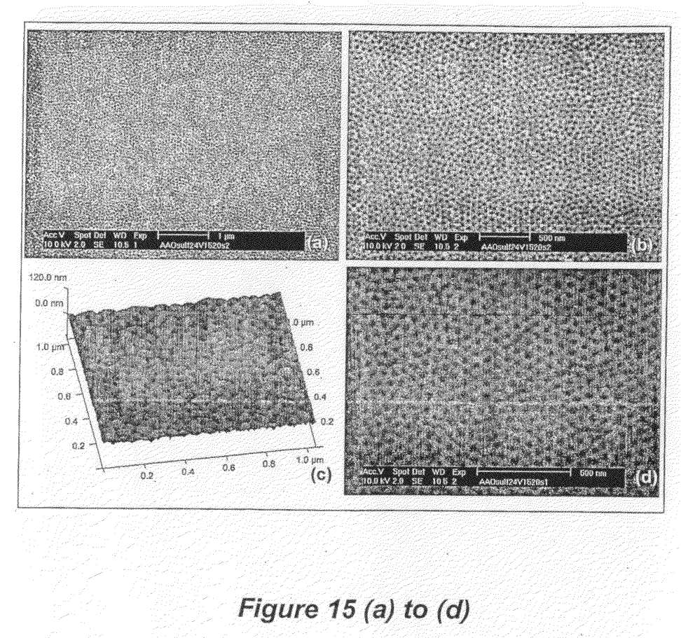

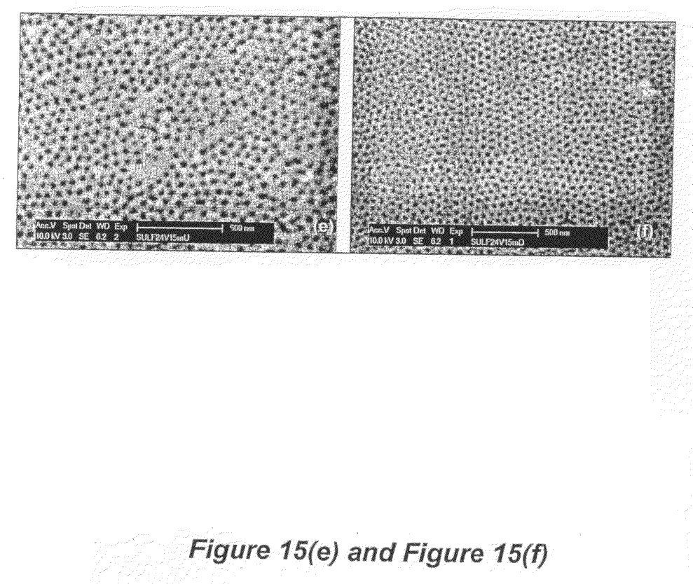

SEM and AFM images of membranes prepared via anodization in sulfuric acid at 24 V are given in FIG. 15. It is evident from these images (and their corresponding scale bars) that anodization of aluminium under these conditions produced relatively smaller nanopores in the alumina film. The final arrangement of pores still follows the characteristic hexagonal pattern with many ordered pore domains apparent. Overall the membrane exhibits a similar degree of ordering to that formed in oxalic acid at 30 V. FIGS. 15a and 15b present SEM images of the top surface of the alumina film depicting the long range order and tightly packed architecture. FIG. 15c is a 3-dimensional representation of the surface topography of the membrane (imaged by AFM) and illustrates the lateral structure of the membrane.

The bottom surface of the prepared film was also characterised by SEM to show the fabrication of a completely nanoporous film. The barrier layer oxide was completely etched in phosphoric acid (0.3 M) for 20 minutes. Again, the topography of the bottom of the AAO film was consistent with its surface counterpart.

Overall, anodization in sulfuric acid at 24 V yielded nanopores with an average pore mouth diameter of approximately 45 nm, and a pore separation (inter-pore distance) of ˜62 nm at the membrane surface. It is proposed that smaller pores are formed under these conditions because of the reduced dissolution of the oxide layer at the lower forming voltage. Under the lower electric field, dissolution at the pore bottom is not as severe as that previously observed (i.e. in oxalic acid at 60 V) and thus results in a smaller sized pore. It has been suggested that anodization in sulfuric acid occurs at a much faster rate which, in conjunction with the slower dissolution, would also contribute to the fabrication of smaller pores.

### Characterisation of AAO Thin Films Prepared in Phosphoric Acid at 60 V

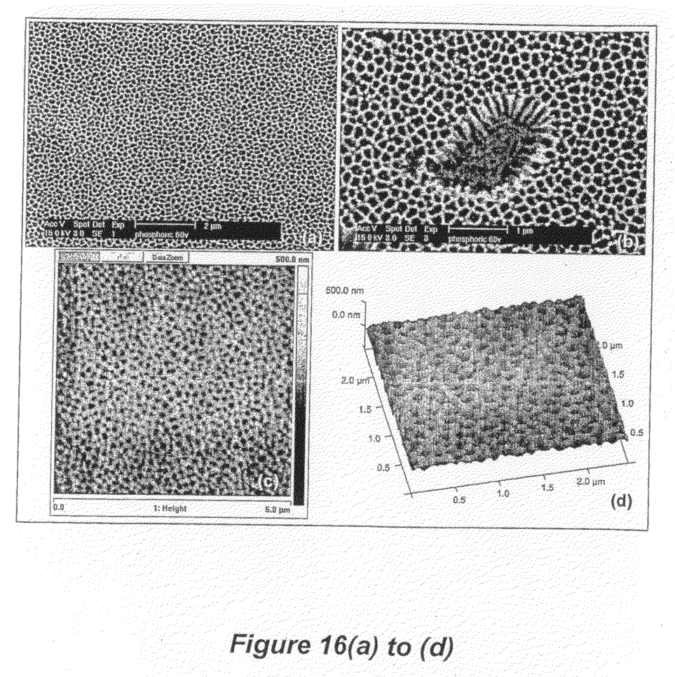

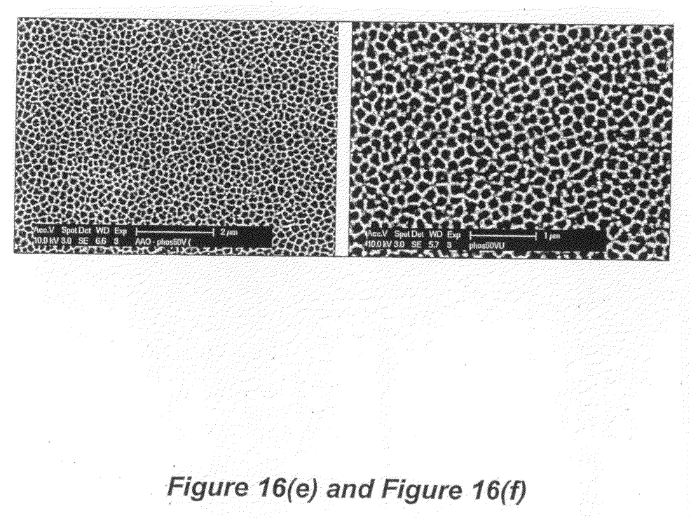

SEM and AFM images of porous alumina films prepared from anodization in phosphoric acid at 60 V are given in FIG. 16. The relatively unstable growth of the aluminium oxide under these conditions resulted in membranes with pore sizes ranging from about 170 nm to about 250 nm. Although the large and mostly non-cylindrical pores deviate from a hexagonal arrangement, it is evident from FIG. 16a that nanoporous architecture is still present across the entire surface of the membrane. The minor defect shown in FIG. 16b shows parallel nano-channels to extend throughout the width of the AAO film.

Overall, the unstable growth and formation of large pores is again due to the high forming voltage. To summarise, it has been shown that the surface charge of the membrane changes from negative to positive at higher voltages and results in an enhanced dissolution of the oxide layer to form the much larger pores.

It is also necessary to explain the use of the more concentrated solution for the preparation. It was originally noted that anodization in phosphoric acid at a similar concentration to that used in the oxalic and sulfuric acid preparations did not produce a significant layer of alumina on the surface; that is, the substrate still appeared to have the shiny, mirrored finish afforded from the electrochemical polishing procedure. Therefore in order to produce a significant porous film in a reasonable time-frame, the concentration of the electrolyte solution was increased.

AFM characterisation was consistent with the SEM images and the 3-dimensional representation of the topography of the AAO membrane illustrates the undulating surface that is expected to influence cell behaviour (FIG. 16d).

### Characterisation of AAO Thin Films Prepared in Tartaric Acid at 200 V

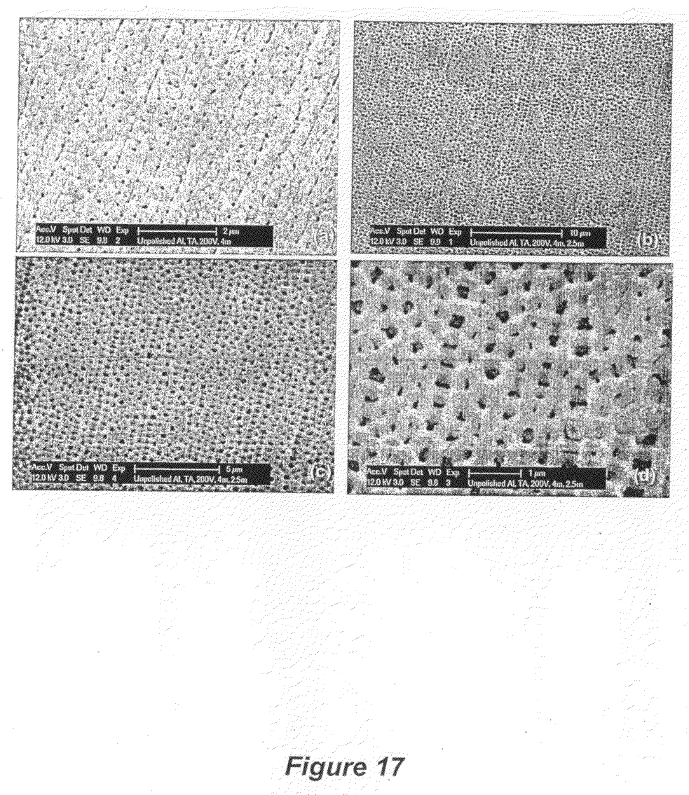

SEM images of porous alumina films prepared from anodization in tartaric acid at 200 V are given in FIG. 17. The enhanced field-assisted dissolution of the anodic film at the significantly higher voltage results in membranes with pore sizes ranging from about 200 nm to about 500 nm.

An obvious advantage of this method when compared to the previous preparations (i.e. at lower voltages in oxalic, sulfuric, and phosphoric acid electrolytes) is the significant reduction in the time of preparation. For example, preparation of an AAO membrane with an inter-pore distance of approximately ˜80 nm (i.e. that formed in oxalic acid at 30 V) requires a primary anodization of 7 hours, and a secondary anodization of 5 hours. However in this case, preliminary results show porous alumina films were prepared from short primary and secondary anodizations of only 4 and 2.5 minutes respectively. This short preparation time may also allow the development of a novel three- or four-step anodization method, potentially increasing pore ordering and regularity over the alumina film.

It is proposed that a similar methodology will apply to the use of malonic acid. It is proposed to try a range of different voltages, including 90V.

Overall, it has been shown that nano-structured MO thin films were successfully prepared from an optimised two step anodization method with a range of pore sizes. It is important to note that the MO films were highly reproducible, as a number of membranes were fabricated for characterisation and skin cell studies.

### Cell Culture

### Preparation of Cells

An epithelial suspension of HaCaT cells was prepared from an existing bank of cells. Cells were enzymatically lifted from tissue culture flasks using a well known method. The existing media was removed from the flask and the cells were washed with PBS [phosphate buffered saline] (10 mL). Trypsin was added to the culture (2-3 mL) and left to stand for 5 minutes in a 37° C./5% CO₂ incubator. DMEM-10% FBS media [fetal bovine serum] (10 mL) was then added to the flask and rinsed up and down with a pipette to remove clumps and resuspend the cells. The culture was transferred to centrifuge tube and spun at 1500 rpm for 5 minutes, after which the supernatant was decanted and the cells resuspended in fresh media. A cell count was then performed and the cell concentration was adjusted with fresh media.

### Keratinocyte Adhesion on AAO Membranes

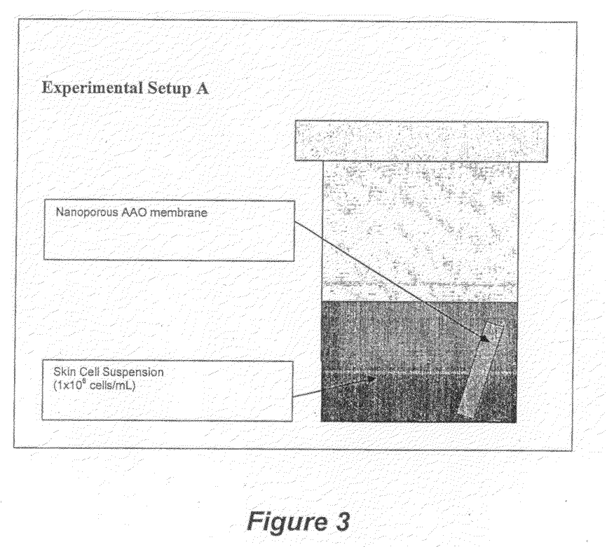

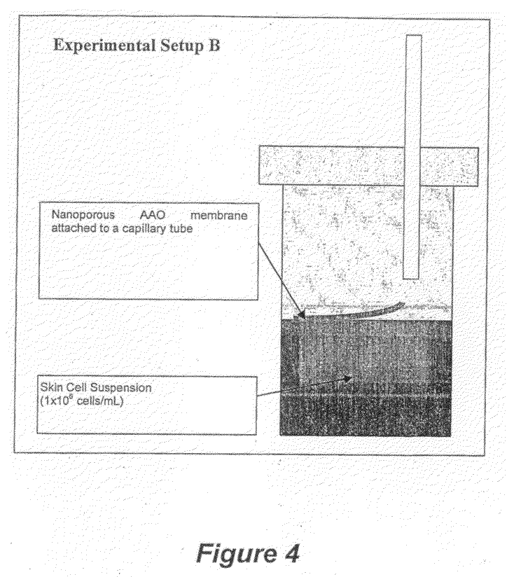

Atomic force microscopy (AFM) was used to qualitatively investigate the adhesion of keratinocytes to the inorganic AAO membrane. Using the experimental setups schematically shown in FIGS. 3 and 4, AAO membranes were brought into contact with HaCaT cell suspension of concentration ˜1×10⁶ cells/mL.

In each experiment, parts of the membrane were removed for imaging after 1 hour and 24 hours of contact. Samples were prepared by mounting the removed portions on AFM steel stubs that had been covered with double sided “3M scotch tape”. The set sample was then washed with fresh media while it was spun by means of a centrifuge to remove any non-adhered cells. The sample was then quickly transferred to the AFM instrument and was imaged over a period of 2-3 hours. AFM scans were performed in ambient laboratory conditions using micro-fabricated silicon cantilevers with silicon nitride sharpened tips in contact mode at a scan rate of 1.52 Hz.

The adhesion of keratinocytes on nanoporous AAO was studied by placing the inorganic substrate in contact with a suspension of freshly cultured skin cells and then imaging the sample with atomic force microscopy (AFM) as described above. Two independent investigations were performed using different experimental setups; (A) AAO membrane completely submersed in skin cell suspension, and (B) AAO membrane floated on the surface of the cell suspension. AFM was employed for these studies as it is a non-destructive technique with the necessary resolution to characterise the underlying nanoporous substrate as well as the cells on its surface.

### Experimental Setup A

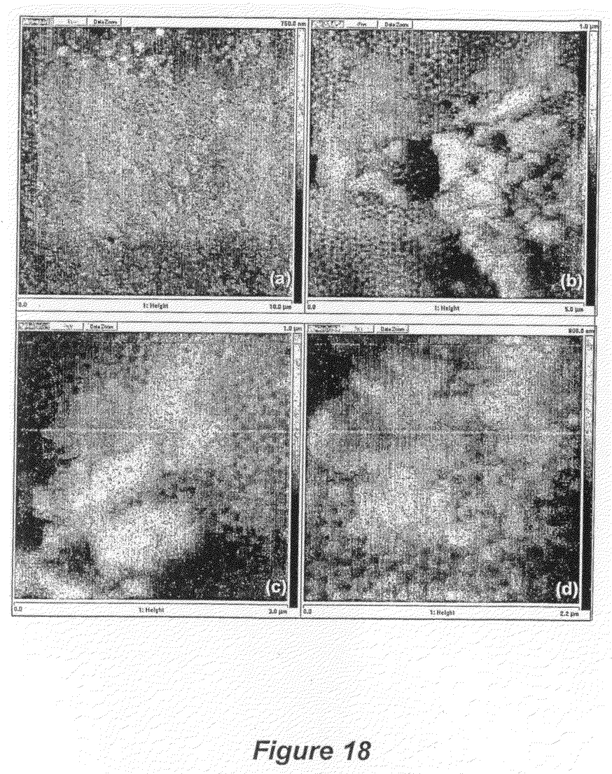

FIG. 18 presents AFM scans of a portion of AAO membrane that had been in contact with skin cell suspension for 1 hour. The cells appear as lighter masses on the darker membrane surface and it is clear from FIG. 18a that keratinocytes had deposited onto the MO substrate to cover a majority of its surface. The higher resolution scans clearly indicate the presence of skin cells on the nanoporous membrane (FIGS. 18b-d). In these images, the highly ordered, hexagonal array of nanopores is evident below the mass of cells on its surface.

Firstly, if keratinocytes were not compatible with nanoporous AAO, they would not readily settle onto its surface and anchor in a manner to that observed. It is important to note that as part of the preparation of the sample for imaging, the membrane was thoroughly washed (while being spun in the centre of a centrifuge) with fresh media to remove any non-adhered cells from the sample. Therefore, it is believed that the cells observed in the AFM scans were firmly anchored to the nanoporous substrate. It is also reasonable to assume that if cell attachment to the membrane were limited, the AFM tip (which was operated in contact mode) would be able to ‘sweep’ them and not record a stable and definite image. The reproducibility of AFM scans at different magnifications strongly supports this assumption, as it would be difficult to capture consistent images if the cells were not firmly attached to the underlying substrate (FIGS. 18b and c). As a result, it can be concluded that cells readily deposit and firmly attach to nanoporous AAO.

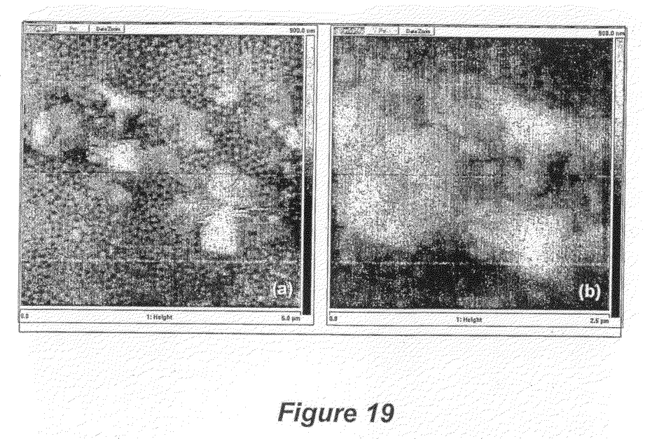

To demonstrate the attachment of cells across the entire surface of the membrane, AFM scans were collected from macroscopically different areas of the sample. It is evident from the high resolution scans of a different area that cells had deposited onto the nanoporous architecture of the AAO membrane (FIG. 19). Consistent features are observable in the magnified image (FIG. 19b), which again suggest firm attachment of cells to the substrate. Overall, it can be concluded that there is a favourable and compatible interaction of human keratinocytes with nanoporous AAO. AFM images have illustrated the deposition and attachment of many cells on the surface of the membrane, which is an encouraging result since cell adhesion determines subsequent cell functions including proliferation and differentiation.

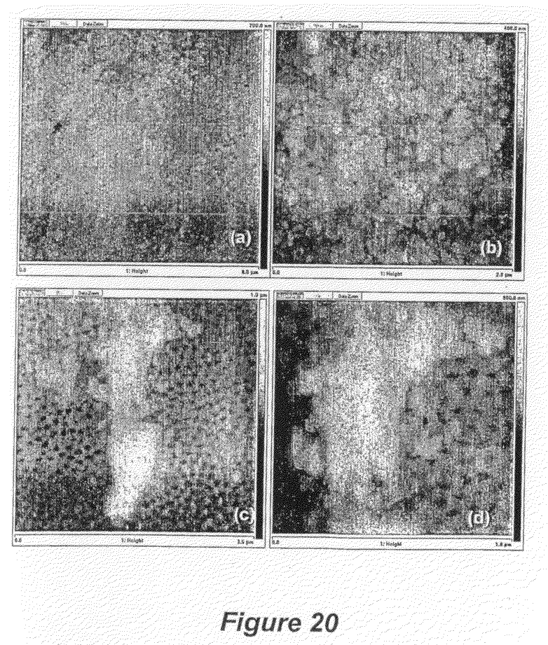

The adhesion of skin cells on anodic aluminium oxide was also studied after 24 hours of contact with the cell suspension. AFM scans of the surface of the membrane at two different areas are given in FIG. 20. Firstly, the images clearly illustrate the existence of skin cells on the surface of the membrane after 24 hours of contact. FIG. 20b highlights the dense coverage of skin cells on the sample, while scans from a different area of the membrane clearly show the presence of cells on the hexagonal array of nanopores (FIGS. 20c & d). Again, the reproducibility of the AFM scans over different magnifications and at different areas of the membrane represents the favourable attachment of cells to the entire surface of the membrane.

The direct observation of keratinocytes on the membrane after the extended period of time is a strong indication of the firm attachment of cells to the non coated, inorganic substrate. As with the previous sample, the membrane was thoroughly washed with fresh media prior to imaging, and so any dead or non-adhered cells would have been removed from the sample. Therefore these results not only confirm cell attachment to nanoporous AAO, but also give an indication of the viability of the cells on the inorganic substrate. It suggests that there is no immediate (i.e. within 24 hours) suicidal or fatal response to the material or the topography of the membrane, which is encouraging for long-term proliferation and its overall application in skin repair.

### Experimental Setup B

A repeat study of the adhesion of keratinocytes on nanoporous AAO was performed to ascertain reproducible results and make specific conclusions about the interaction of cells with the membrane surface. In this experimental setup, the membrane was placed in contact with the cell suspension only at its surface, to observe if cells preferentially adhered to the membrane surface, or just deposited on the substrate due to gravity.

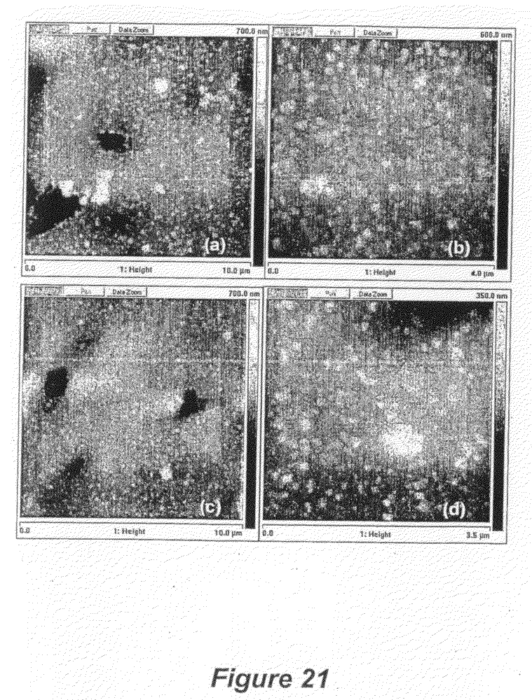

The images in FIG. 21 are AFM scans of the membrane surface at two independent areas of the sample after 1 hour of contact with the skin cell suspension. Skin cells were observable in all of the AFM scans (over different magnifications) highlighting and confirming the favourable attachment of keratinocytes to the surface of nanoporous AAO (FIG. 21). In this study, the observation of skin cells on the alumina film was an intriguing result since the membrane was only in contact with the surface of the cell suspension. Therefore, it is reasonable to assume that cell attachment to the membrane was spontaneous and was not forced under gravity. An explanation for this is that the adherence of biological material to AAO thin films is due to the inherent surface-charge, chemical and nanotopography of the substrate. The accumulation of charged crystalline lattice and electronic defects at the surface leads to a net dipole moment that can extend over several nanometres. Consequently, when biological material comes into contact with the membrane, it can adhere to the surface. The observation of skin cells on the substrate verifies that keratinocytes readily deposit and in-fact attach to nanoporous AAO.

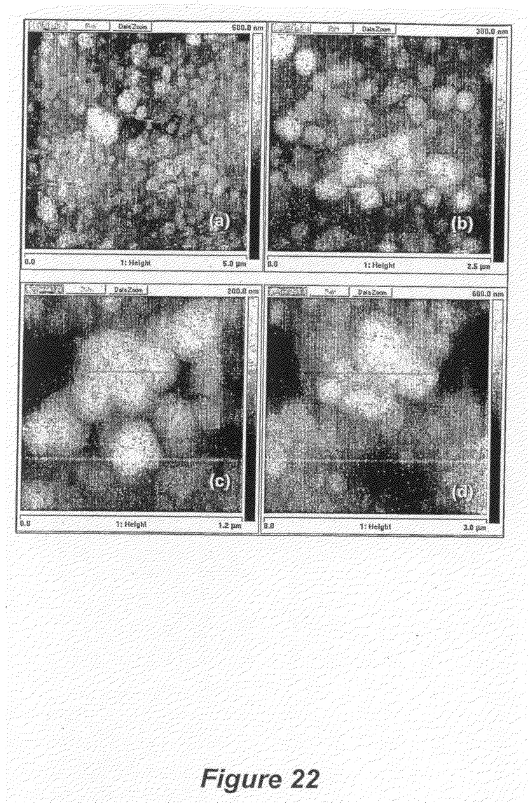

As with the previous investigation, a portion of the membrane was also imaged after 24 hours of cell contact. FIG. 22 presents AFM scans of the sample at various magnifications and different areas of the membrane surface. Again, there was a significant coverage of cells across the entire surface of the inorganic substrate over the longer time-frame. The interesting feature in these images was the observation of larger collections of cells on the surface, as it is suggestive of cell interaction and proliferation on the nanoporous substrate (FIGS. 22b & c). FIG. 22d is an AFM scan of a substantially larger accumulation of cells at a different area on the membrane, supporting the notion of further cell function at the membrane surface.

It is understandable that the majority of the cells in the suspension would have settled to the bottom of the culture flask after 24 hours. Therefore, it can be assumed that the cells observed in the AFM scans were those which had initially attached to the membrane at first contact. It is also expected that if the cells had died from contact with the alumina film they would detach from the surface, and be washed away during sample preparation. As a result, the observation of a large number of cells on the inverted substrate over 24 hours gives a clear indication that cells were anchored and viable on the nanoporous membrane, which is promising for further cell function on the substrate.

Overall, it can be strongly concluded that cells readily deposit and firmly attach to inorganic, nanoporous AAO membranes. Consistent AFM scans from several experiments, over different areas and different magnifications of the membrane have proven the favourable deposition and attachment of human keratinocytes on AAO.

### Real-Time Microscopy of Keratinocytes on Nanoporous AAO

To this point, adhesion studies of keratinocytes on AAO films by AFM have proven the attachment of cells to the nanoporous membrane, but have only given tentative information about the viability and proliferative function of skin cells on the membrane. It is obvious that if cells eventually die or do not readily proliferate due to contact with the inorganic substrate there is no potential use in skin repair. As a result, real-time microscopy was employed to record cell behaviour on nanoporous AAO and thus give an indication of the growth potential on the substrate.

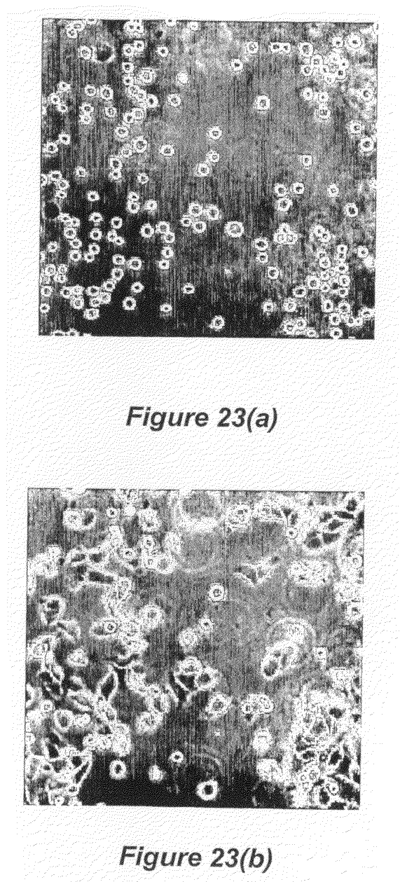

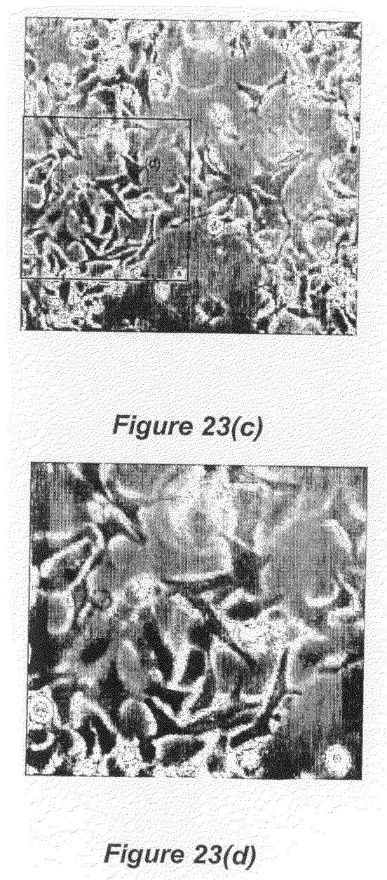

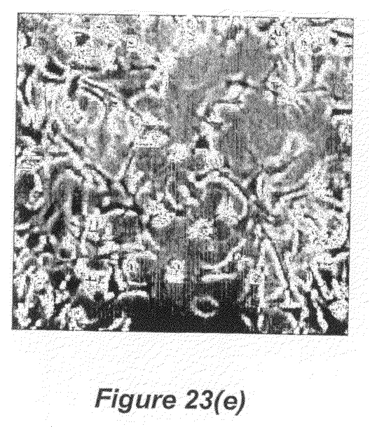

FIG. 23 presents screen captures of the first 24 hours of cell contact with an AAO thin film. Firstly, FIG. 23a is an image of the sample at time 0 —that is, just after the cells were added to the membrane surface and the sample chamber was prepared. At this initial stage, the keratinocytes were round in shape, which indicated that they had not yet attached to the membrane. When cells are enzymatically removed from a culture vessel by trypsin, they condense into the observed round shape before settling back into their inherent form when they attach to a substrate. FIG. 23b illustrates the deposition and attachment of keratinocytes to the membrane after ˜4 hours. At this time it was observed that the majority of cells had transformed into their natural rectangular shape, which is indicative of adhesion on the underlying substrate.

Once the cells had attached to the substrate and settled back into their inherent form, cell division and proliferation was witnessed across the surface of the AAO membrane. FIG. 23c presents a screen capture of the sample after 12 hours. The image clearly indicates the interaction and division of cells on the inorganic membrane, with no indication of abnormal cell function. The enlargement of the specified area given in FIG. 23d illustrates normal cell shape and definite cell interaction. When FIGS. 23 c & d are compared with FIG. 23a, it is clear that cell division and proliferation had occurred on the surface of the membrane.

FIG. 23e characterises cell behaviour after 24 hours of sample preparation. Again, from the direct comparison to FIG. 23c, it is clear that significant cell proliferation had occurred on the nanoporous AAO membrane. It can be seen that there was noticeably more coverage of the membrane surface with skin cells, which confirms cell division and viability on the membrane over 24 hours. This result is consistent with the AFM data collected, which also indicated a dense coverage and larger accumulations of cells on the surface after 24 hours. A complete time-lapse movie showing ˜30 hours of the experiment can be accessed electronically in Appendix A (mov format).

The keratinocytes were still viable on the nanoporous substrate after 72 hours. Although the results are not presented here, the observation of live cells on the membrane after this time-frame was important, as it confirmed original conclusions of cell compatibility with the inorganic substrate.

This is the first time cell adhesion and proliferation has been observed on nanoporous AAO. There is no literature available which characterises epithelial cell behaviour on the surface of anodic alumina.

### Keratinocyte Viability/Proliferation on AAO Membranes

In order to observe the viability and proliferation of skin cells on nanoporous anodic aluminium oxide, in-situ, real-time microscopy imaging was performed. The sample chamber of the microscope was prepared by mounting an AAO membrane [prepared from anodization in phosphoric acid at 60V] on a glass slide and adding an aliquot of live skin cell suspension to the membrane (200 μL, 1×10⁶ cells/mL). Cells were allowed to superficially adhere to the membrane for 10 minutes and then the chamber was sealed with fresh media. The sample chamber was mounted on a temperature controlled stage (37° C.) and cell behaviour was recorded over a period of 72 hours at 20×magnification using a camcorder attached to the microscope. The living culture, time-lapse movie was saved and converted using the available software at the imaging facility.

### Proliferation Assay of Keratinocytes on AAO Membranes

Once it had been established that keratinocytes readily adhered and proliferated on nanoporous AAO, exhibiting no unfavourable response to the inorganic material or its nanoporous topography over a significant period of time (72 hours), it was necessary to begin investigations into the effect of nano-architecture on cell behaviour. Ultimately, the aim of this study was to design a template that optimises the behaviour of keratinocytes to reduce the time to heal and enhance wound repair, and the next step was to perform a proliferation assay to quantify the growth of cells across the different nanoporous membranes.

The technique is a colorimetric method that utilises a novel tetrazolium compound [3-(4,5-dimethylthiazol-2-yl)-5-(3-carboxymethoxy-phenyl)-2-(4-sulfophenyl)-2H-tetrazolium, inner salt; MTS] that is bio-reduced by cells into a coloured formazan product. The conversion is presumably accomplished by NADPH or NADH produced by dehydrogenase enzymes in metabolically active cells. The quantity of formazan product as measured by its absorbance at 492 nm is directly proportional to the mitochondrial activity of cells, which in turn is related to the number of living cells in culture (Technical Bulletin, Promega, 2005).

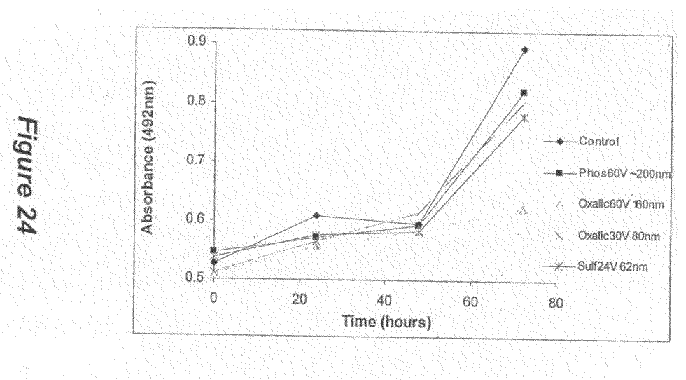

FIG. 24 presents the results of a 3-day proliferation assay, which suggested there was an effect of nano-architecture on cell growth. From the graph, it can be seen that there were significantly fewer cells in the culture well containing the AAO membrane with 160 nm pore separation after the 3 day assay (i.e. AAO membrane prepared in oxalic acid at 60 V), when compared to the control, and the wells containing the other AAO membranes. A linear discriminate analysis confirmed the existence of two distinct groups (those indicated in FIG. 24), with 100% separation at 95% confidence. It is premature to state, without confirmatory evidence, that cell growth is retarded by the topography of the membrane prepared in oxalic acid at 60 V. However, it is obvious that there was a significant difference in the proliferation of keratinocytes on this membrane, which justifies additional research into the effects of nanoporous topography on keratinocyte behaviour.

It is also interesting that the different result was obtained from a membrane that had a pore size in the middle range of that prepared. It was initially expected that proliferation would either increase or decrease with pore size, yet this result proposes otherwise. Again further research is necessary to confirm this result.

It is important to note that a repeat assay was performed in triplicate, over a period of 4 days (including day 0). However, because of the pre-treatment of the cell line before the assay (cells were serum starved to regulate cell growth from the same position in the cell cycle), and the difficulty of cutting the membranes to cover the wells of a much smaller culture plate, the results obtained were incomparable and unstable across the 4 days. The controls also displayed volatile results (% RSD of 27% at day 4) and the experiment was deemed unsuccessful. It is proposed that future experiments will be performed under the same conditions as the first assay to obtain confirmatory results.

In order to quantify the proliferation of cell growth on each of the different membranes, a standard proliferation assay was performed (Promega 2005). Keratinocyte proliferation was investigated on each of the nanoporous AAO membranes over 3 days of culture (including day 0). Initially, a 24-well culture plate was prepared by cutting each of the membranes to cover the bottom of the wells.

Freshly suspended cells were seeded onto the alumina substrates (400 μL of ˜2.5×10⁴ cells/mL in media; to give ˜10,000 cells per well) and allowed to adhere in a 37° C., humidified, 5% CO₂ incubator for 4 hours. MTS (80 μL) was added to each of the culture wells for the ‘Day 0’ reading and returned to incubate for 4 hours. The supernatant (200 μL) from each ‘Day 0’ well was carefully removed and transferred to a 96-well culture plate for measurement.

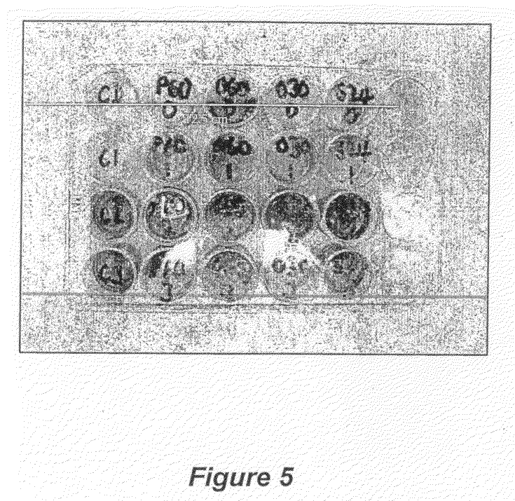

The absorbance of the culture media was measured at 492 nm with an ‘ELISA 96-well automatic plate reader’. The original 24 well-plate was returned to the incubator (37° C./5% CO₂) and the procedure was repeated (from the addition of MTS) at the same time for each consecutive day (for days 1-3). Controls (i.e. with no nano-membrane) were also conducted for each day and were subjected to the same protocol as all other wells. FIG. 5 illustrates a digital photograph of the experimental setup for the proliferation assay.

Overall, these preliminary results are very encouraging for the potential exploitation of AAO topography to positively influence epithelial cell behaviour. Not only does the assay confirm the viability and the presence of live cells on the membrane after 72 hours, but also indicated that surface topography may have an effect on cell proliferation.

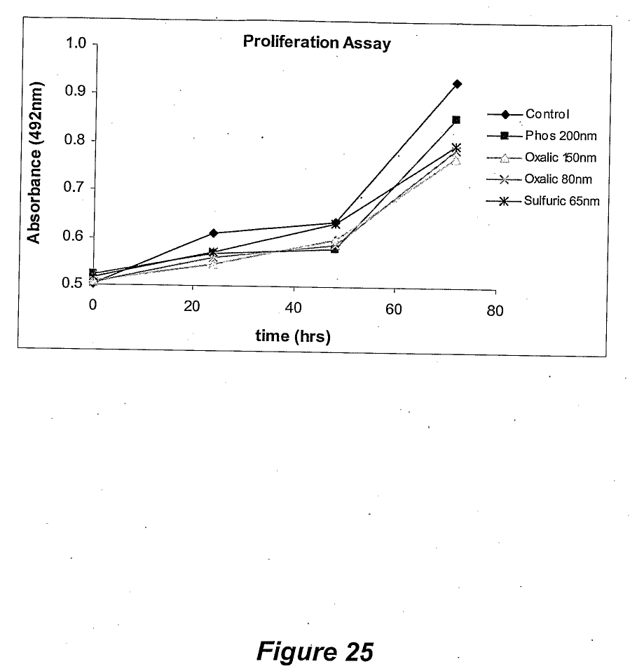

A further 3 day proliferation assay of keratinocytes on AAO films was carried out, in duplicate, to again investigate the effect of nano-architecture on cell behaviour. The study was conducted as described above using AAO films with inter pore distances of 200 nm, 150 nm, 80 nm and 65 nm. The results of this further study are shown in FIG. 25. The quantity of Formazan product produced by the cells as measured by the absorbance at 492 nm is directly proportional to the number of living cells in culture. As evident from the graph, there seems to be an effect on cell behaviour at about 200 nm.

From the data presented to date, it appears that nano-architecture under 100 nm may be too small for there to be any significant impact on cell behaviour, perhaps being perceived as a flat surface by the relatively larger cells. However, as the pore geometry approaches approximately 200 nm, there seems to be some effect on the response of the skin cells when compared to those below 100 nm (see FIG. 25). The lowest proliferation of cells was observed at 150 nm (a similar distance to 160 nm, as seen in the previous assay). Therefore these results appear to suggest that the behaviour of cells is sensitive somewhere in this region of nano-pore distance between 200 nm and about 100 nm, although further assays would be required to confirm that inter pore distance of less than 100 nm does not affect, for example, apoptosis or differentiation. Further work will concentrate on the area, and on increasing the pore size range above 200 nm.

Although the invention has been described with reference to certain preferred embodiments, it will be appreciated that many variations and modifications may be made within the scope of the broad principles of the invention. Hence, it is intended that the preferred embodiments and all of such variations and modifications be included within the scope and spirit of the invention.

1. A nanoporous membrane structure characterised in that it has a hexagonal array of tubes which open with pores wherein there is a substantially uniform inter-pore distance between the tubes and wherein the distance is within the range from 10-500 nm and wherein the tubes have a depth of up to 500 μm.

2. A nanoporous membrane structure according to claim 1 wherein the tubes are aligned substantially perpendicular to the surface of the membrane.

3. A nanoporous membrane structure according to claim 1 wherein the density of the pores is between the range from 10⁹ to 10¹² pores/cm².

4. A nanoporous membrane structure according to claim 1 wherein the inter-pore distance is within the range from 50 to 350 nm.

5. A nanoporous membrane structure according to claim 1 wherein the inter-pore distance gradually decreases from the periphery of the membrane to the centre of the membrane.

6. A nanoporous membrane structure according to claim 1 wherein the inter-pore distance gradually increases from the periphery of the membrane to the centre of the membrane.

7. A nanoporous membrane structure according to claim 1 wherein the membrane comprises a hexagonal array of tubes with a substantially uniform inter-pore distance towards the centre of the membrane but a different uniform inter-pore distance towards periphery of the membrane.

8. A method for preparing a nanoporous membrane, comprising the steps of:
(a) preparing an aluminium film;
(b) subjecting the aluminium film to a first anodising step;
(c) subjecting the aluminium film to wet chemical etching;
(d) subjecting the aluminium film to a second anodising step, wherein the conditions employed in this step are similar to or substantially the same as the conditions as used in the first anodising step.

9. A method according to claim 8 wherein the first anodising step is carried out using a constant voltage and an aqueous solution of an acid.

10. A method according to claim 9 wherein the constant voltage ranges from between about 15 to about 200 volts.

11. A method according to claim 10 wherein the constant voltage is selected from 24 volts, 30 volts, 60 volts or 200 volts.

12. A method according to claim 9 wherein the aqueous acid solution is selected from sulphuric acid, oxalic acid, phosphoric acid, chromic acid, tartaric acid and malonic acid.

13. A method according to claim 9 wherein the voltage is 30 V and the aqueous acid solution is oxalic acid.

14. A method according to claim 9 wherein the voltage is 60 V and the aqueous acid solution is oxalic acid.

15. A method according to claim 9 wherein the voltage is 24 V and the aqueous acid solution is sulphuric acid.

16. A method according to claim 9 wherein the voltage is 60 V and the aqueous acid solution is phosphoric acid.

17. A method according to claim 9 wherein the voltage is 200 V and the aqueous acid solution is tartaric acid.

18. A method according to claim 9 wherein the voltage is 90 V and the aqueous acid solution is malonic acid.

19. A method according to claim 8 A method according to any one of claims 8 to 18 wherein the first anodising step is carried out at a temperature from 0° C. to room temperature.

20. A method according to claim 8 further comprising the steps of:
(e) contacting the aluminium film from step a with a material capable of supporting the film;
(f) suspending the aluminium film in a solution which completely removes the unprotected aluminium substrate; and
(g) removing the layer of oxide to produce nano-channels throughout the alumina film.

21. A method according to claim 8 wherein the aluminium film of step (a) has a protective layer applied to one side of the aluminium film prior to the first anodising step of step (c).

22. A nanoporous membrane structure produced by the method of claim 8.

23. A method of preparing a nanoporous membrane structure coated with viable cells comprising the steps of contacting a nanoporous membrane structure with a suspension of cells for a period of time sufficient to deposit cells onto the membrane.

24. A method for preparing a homogenous population of cells, wherein the method comprises the steps of:
(a) manufacturing a nanoporous membrane structure;
(b) growing cells on the nanoporous membrane structure; and
(c) harvesting the cells from the membrane.

25. A method of treating a patient in need of tissue damage repair, said method comprising the steps of:
(a) manufacturing a nanoporous membrane structure;
(b) growing cells on the nanoporous membrane structure; and
(c) applying cell coated nanoporous membrane structure to a wound.

26. A method of treating a patient in need of tissue damage repair, said method comprising the steps of:
(a) manufacturing a nanoporous membrane structure;
(b) contacting the nanoporous membrane structure with a solution of cultured cells; and
(c) applying the nanoporous membrane structure coated with ceils to the site of tissue damage.
